(19) United States
(12) Patent Application Publication
(10) Pub. No.: US 2021/0275098 A1
(43) Pub. Date: Sep. 9, 2021

(54) METHODS AND DEVICES FOR INFORMATION ACQUISITION, DETECTION, AND APPLICATION OF FOOT GESTURES

(71) Applicant: XIN TIAN, Niskayuna, NY (US)

(72) Inventor: XIN TIAN, Niskayuna, NY (US)

(21) Appl. No.: 16/332,756

(22) PCT Filed: Sep. 13, 2017
(86) PCT No.: PCT/US17/51431

§ 371 Date: Mar. 12, 2019

Related U.S. Application Data
(63) Continuation of application No. 15/331,410, filed on Oct. 21, 2016, now abandoned.
Application No. 15/331,410 is a continuation of application No. 15/283,764, filed on Oct. 3, 2016, now Pat. No. 10,448,892.
(60) Provisional application No. 62/483,966, filed on Apr. 11, 2017.
(60) Provisional application No. 62/470,848, filed on Mar. 13, 2017.
(60) Provisional application No. 62/394,048, filed on Sep. 13, 2016.
(60) Provisional application No. 62/394,048, filed on Sep. 13, 2016.

(51) Int. Cl. A61B 5/00; A43B 3/00; A61B 5/0205; G01C 17/00; A61B 5/103

(52) U.S. Cl. CPC A61B 5/6807; A43B 3/0005; A61B 5/0205; A61B 5/1112; A61B 5/1038; A61B 5/7455; A61B 2562/0219; G01C 17/00

(57) ABSTRACT

Methods and devices for information acquisition/detection/application of foot gestures are provided. A method includes acquiring information related to foot gesture features, and sending the acquired information related to the foot gesture features to an electronic device for a foot gesture detection. The foot gesture features includes one or more of a foot pointing direction of each of a user's one foot or both feet, a foot touch state of a of user's one foot, and a foot touch state of a user's both feet. The foot touch state is determined based on whether one or multiple parts of a user's foot sole touch or press a supporting platform, the supporting platform including the ground.

### CROSS-REFERENCES TO RELATED APPLICATIONS

This application claims the priority of U.S. Provisional Patent Application Nos. 62/483,966, filed on Apr. 11, 2017, and 62/470,848, filed on Mar. 13, 2017; and U.S. patent application Ser. No. 15/331,410, filed on Oct. 21, 2016, and Ser. No. 15/283,764, filed on Oct. 3, 2016, which claim priority to U.S. Provisional Patent Application No. 62/394,048, filed on Sep. 13, 2016, the entire content of all of which is incorporated herein by their reference.

### FIELD OF THE DISCLOSURE

The present disclosure generally relates to the field of information technology and electronic systems, more particularly, relates to methods and devices for information acquisition, detection and application of foot gestures.

### BACKGROUND

Information collected from a user's foot or both feet by various sensing devices is mostly used to measure physical activities of the user in various health applications, e.g., health applications on smart phones. However, the functions of detecting user foot gestures and using user foot gestures for device control and user-device/user-application interactions are not adequately developed. Currently, the control of various electronic devices, e.g., computers, smart phones, game consoles, and user-device/application interactions supported by the devices are predominantly hand-based. Common examples of input devices and device components supporting handed-based controls of various electronic devices include keyboard, mouse, joy stick, touch screen/pad, multi-touch screen/pad, etc.

In particular, input devices/device components supporting multi-touch detections, e.g., multi-touch screen and multi-touch pad, are able to obtain user touch point coordinate information and touch point movement information. The information then is used to support hand gesture based device/application control and user-device/application interactions. Corresponding to multi-touch input devices and hand gesture detection technologies, this invention describes a complete set of solutions for the detection of various user foot gestures and foot gesture based device/application control and device/application interactions. The set of solutions further include i) user foot gesture feature information acquisition device and corresponding methods, and ii) the method for the detection of user foot gestures using foot gesture feature information, and devices using the methods to achieve foot gesture detection and foot gesture based user-device/application interactions.

Corresponding to coordinates of a user's hand touch points which are the key information supporting hand gesture detections, foot gesture features supporting foot gesture detection include foot pointing direction(s) of a user's one foot or both feet, and foot touch states determined by the touch state of multiple touch areas of a user's sole to the ground or any support surface. Additional foot gesture features also include foot tilt angle(s) from a user's one foot or both feet and various foot moving trajectory state related features. Corresponding to multi-touch screen or multi-touch pad which is an input device supporting user hand gesture detections, the foot gesture feature information acquisition device is configured to providing information related to various foot gesture features from a user's foot or both feet. An embodiment of the foot gesture feature information acquisition device is a compass embedded footwear system disclosed in the present invention.

### BRIEF SUMMARY OF THE DISCLOSURE

One aspect of present disclosure provides a method, including acquiring information related to foot gesture features, and sending the acquired information related to the foot gesture features to an electronic device for a foot gesture detection. The foot gesture features includes one or more of a foot pointing direction of each of a user's one foot or both feet, a foot touch state of a of user's one foot, and a foot touch state of a user's both feet. The foot touch state is determined based on whether one or multiple parts of a user's foot sole touch or press a supporting platform, the supporting platform including the ground.

Another aspect of present disclosure provides a method, including: receiving information related to foot gesture features from an information acquisition device; obtaining the foot gesture features using the received information; detecting a foot gesture using the obtained foot gesture features; and generating a control signal based on the detected foot gesture.

Another aspect of present disclosure provides an information acquisition device. The information acquisition device includes an information acquisition member and a communication member. The information acquisition member is configured to acquire information related to foot gesture features, the foot gesture features including one or more of a foot pointing direction of each of a user's one foot or both feet, a foot touch state of a of user's one foot, and a foot touch state of a user's both feet. The foot touch state is determined based on whether one or multiple parts of a user's foot sole touch or press a supporting platform, the supporting platform including the ground. The communication member is configured to send the acquired information related to the foot gesture features to an electronic device for a foot gesture detection.

Another aspect of present disclosure provides an electronic device. The electronic device includes an information receiver, a foot gesture detector, and a command generator. The information receiver is configured to receive information related to foot gesture features from an information acquisition device. The foot gesture detector is configured to obtain the foot gesture features using the received information and detect a foot gesture using the obtained foot gesture features. The command generator is configured to generate a control signal based on the detected foot gesture.

A non-transitory computer readable storage medium, storing computer-executable instructions for, when being executed, one or more processors to perform the disclosed methods.

### BRIEF DESCRIPTION OF THE DRAWINGS

The following drawings are merely examples for illustrative purposes according to various disclosed embodiments and are not intended to limit the scope of the present disclosure.

FIG. 1 illustrates an exemplary compass-sensor embedded footwear system according to various embodiments of the present disclosure, which is an embodiment of the foot gesture feature information acquisition device;

FIG. 2 illustrates an exemplary control-communication unit used in the compass-sensor embedded footwear system in FIG. 1 according to various embodiments of the present disclosure;

FIG. 3 illustrates another exemplary compass-sensor embedded footwear system according to various embodiments of the present disclosure;

FIG. 4 illustrates an exemplary power-control-communication unit used in the compass-sensor embedded footwear system in FIG. 3 according to various embodiments of the present disclosure;

FIG. 5 illustrates another exemplary compass-sensor embedded footwear system according to various embodiments of the present disclosure;

FIG. 6 illustrates an exemplary power-control-communication-compass unit used in the compass-sensor embedded footwear system in FIG. 5 according to various embodiments of the present disclosure;

FIG. 7 illustrates an exemplary arrangement of pressure sensors at designed sole areas according to various embodiments of the present disclosure;

FIG. 8 illustrates an exemplary relationship between a user's local North-East (N-E) coordinate system, a compass sensor's own reference X-Y 2 dimensional (2D) coordinate system, and a foot direction vector according to various embodiments of the present disclosure;

FIG. 9 illustrates exemplary left and right footwear measurement-information sets in a compass-sensor embedded footwear system at data sampling times according to various embodiments of the present disclosure;

FIG. 10 illustrates processing of pressure sensor measurements to obtain sole area touch detection results, foot-level touch detection results, i.e., single foot touch states, and a user touch detection outcome, i.e., Bi-foot foot touch state, at a data sampling time according to various embodiments of the present disclosure;

FIG. 11 illustrates a user forward direction vector V_{FWD} obtained by fusing foot direction vectors from both feet according to various embodiments of the present disclosure;

FIG. 12 illustrates joint processing and fusion of left and right foot direction vectors as well as the pressure sensor measurements to derive a user forward direction vector V_{FWD} according to various embodiments of the present disclosure;

FIG. 13 illustrates an information processing flow at a data sampling time in a compass-sensor embedded footwear system according to various embodiments of the present disclosure;

FIG. 14 illustrates an exemplary system operation configuration including a compass-sensor embedded footwear system and an (external) electronic device according to various embodiments of the present disclosure;

FIG. 15 illustrates another exemplary system operation configuration including a compass-sensor embedded footwear system and an (external) electronic device according to various embodiments of the present disclosure;

FIG. 16 illustrates another exemplary compass-sensor embedded footwear system according to various embodiments of the present disclosure; and

FIG. 17 illustrates another exemplary compass-sensor embedded footwear system and an artificial reference magnetic field created by magnetic source(s) according to various embodiments of the present disclosure.

FIG. 18 illustrate Bi-foot touch states corresponding to different touch areas touching (pressing) the ground

FIG. 19 illustrate left and right foot pointing directions in reference to a (fixed) local North direction

FIG. 20 illustrates the left foot directed Tapdown gestures

FIG. 21 illustrates the right foot directed Tapdown gestures

FIG. 22 illustrate a set of Bi-Foot touch-only gestures that is capable of replacing the function of for direction buttons for up, down, left and right control

FIG. 23 illustrate a set of Bi-Foot directed Tapdown gestures that is capable of replacing the function of a joystick

FIG. 24 shows the processing flow for the detection of a foot gesture

FIG. 25 illustrates the concept of foot tilt angles

FIG. 26 illustrates the evaluation of foot tilt angle using measurements from a 3-axis accelerometer in the compass sensor unit 105/205

FIG. 27 illustrates the use of directed Tapdown foot gesture for acceleration and brake control in a driving game with foot tilt angle information as an additional gesture “strength” parameter

FIG. 28 illustrates the relationships between foot tilt angle γ_{L}/γ_{R} (1001/1002), the original 2D foot pointing direction vector V_{LF}/V_{RF} (701/702) and a 3D foot pointing direction V_{LF3D}/V_{RF3D}(1003/1004) in a local stationary (fixed/non-rotating) 3D coordinate system

FIG. 29 illustrates the relationship between the variation/change in foot tilt angle γ_{L}/γ_{R} (1001/1002) and the rotation angle around the gyro sensor's x-axis in an assumed sensor placement configuration

FIG. 30 shows the processing flow at a sampling time for the estimation of foot tilt angle γ_{L}/γ_{R} (1001/1002) when a user's foot is moving or stationary.

FIG. 31 illustrates the relationship between the variation/change in 3D foot pointing direction (1003/1004) and the rotation angle around the gyro sensor's Z-axis in an assumed sensor placement configuration

FIG. 32 shows the processing flow at a sampling time for the derivation (estimation) of foot pointing direction information, e.g., foot pointing direction vector V_{LF}/V_{RF} (701/702) or foot pointing angle ωL/ωR (707/708), when a user's foot is moving or stationary.

FIG. 33 illustrates the concepts of 3D foot moving trajectory (1008/1009) and foot moving trajectory states.

FIG. 34 shows the processing flow at a sampling time, for the estimation of foot moving trajectory (states).

### DETAILED DESCRIPTION

This invention describes a complete set of methods and devices for i) the acquisition of various user foot gesture feature information, ii) the detection of various user foot gestures using a range of foot gesture features, and iii) supporting foot gesture based user device/application control/interactions in electronic devices. Corresponding to coordinates of a user's hand touch points being key inputs supporting hand gesture detections, information on a range of foot gesture features are used to support the detection of various foot gestures. These foot gesture features include two basic/fundamental types of foot gesture features which are the pointing direction(s) of a user's one foot or both feet, and foot touch states determined by the touch state of multiple touch areas of a user's sole(s) to the ground or any support surface. Additional foot gesture features also include foot tilt angle(s) from a user's one foot or both feet and various foot moving trajectory state related features. Corresponding to multi-touch screen or multi-touch pad as the key input device supporting user hand gesture detections, a foot gesture feature information acquisition device is used for obtaining information related to various user foot gesture features including user foot pointing directions, user foot touch states and additional foot gesture features including user foot tilt angles, user foot moving trajectory state related features, etc.

Methods and devices disclosed in this invention support the detection of various user foot gestures and foot gesture based device/application control and interaction in various electronic devices including computers, smart phones, game consoles, virtual reality devices, etc.

As one embodiment of the foot gesture feature information acquisition device, a compass-sensor embedded footwear system is disclosed. Various foot gestures, foot gesture features and other concepts mentioned above related to foot gesture detections are defined and explained in details along with the compass-sensor embedded footwear system.

Reference will now be made in detail to exemplary embodiments of the disclosure, which are illustrated in the accompanying drawings. Hereinafter, embodiments consistent with the disclosure will be described with reference to drawings. Wherever possible, the same reference numbers will be used throughout the drawings to refer to the same or like parts. It is apparent that the described embodiments are some but not all of the embodiments of the present disclosure. Based on the disclosed embodiment, persons of ordinary skill in the art may derive other embodiments consistent with the present disclosure, all of which are within the scope of the present disclosure.

As an embodiment of the foot gesture feature information acquisition device, the present disclosure provides a compass-sensor embedded footwear system and operation method thereof to achieve its functions as a foot gesture feature information acquisition device. An exemplary compass-sensor embedded footwear system may include a footwear and/or a pair of footwear corresponding to a pair of human feet including, e.g., a left footwear and a right footwear. Each footwear includes a compass sensor, (i.e., a sensor that is able to provide direction/direction angle measurements of the North direction in its own reference 2D coordinate system) to provide foot directional information; two pressure sensors to obtain pressure measurements at designed sole areas of the footwear to obtain user foot touch state, and/or a control-communication unit and a power module supporting the system operation and distribution of obtained information related to foot gesture features to other electronic devices.

In some embodiments, the footwear used in the compass-sensor embedded footwear system may include a footwear sole and footwear body. In other embodiments, the footwear used in the compass-sensor embedded footwear system may be only a footwear sole such as a footwear insole. For example, the footwear may be a shoe having a shoe sole (footwear sole) and a shoe body (footwear body). In another example, the footwear may be a footwear sole such as a shoe insole, which is a separate layer that can be placed in any shoes.

By using the disclosed compass-sensor embedded footwear system, information related to various gesture features (including foot gesture feature information and information used to derive foot gesture features) can be obtained, and distributed to an electronic device, such as computer, smart phone, game console, etc. Using information from the compass-sensor embedded footwear system, foot gesture features include foot pointing directions, foot touch, and other foot gesture features including foot tilt angle, foot moving trajectory state related features, of a person wearing the footwear, may be obtained with or without further processing at an electronic device.

According to the present disclosure, in one embodiment, foot and/or user directional information and a range of foot gesture information from a user's feet may be effectively provided to a device/devices such as smart phones, tablets, game consoles, computers, to achieve natural hand-free user experiences for navigation in simulated virtual world, for example, in gamming applications and other types of applications. In one embodiment, products based on the present disclosure may be a new type of foot-wearing input device for computers, smart phones, tablet, game console, etc.

As used herein, the term “foot directional information” refers to direction(s) that foot/feet in operation point at. The term “foot directional information” and “foot pointing information” may be interchangeably used in the present disclosure.

As used herein, the term “foot gestures” may include simple gestures, such as taps by foot/feet, and complex gesture behaviors, such as walking, jumping, running, etc.

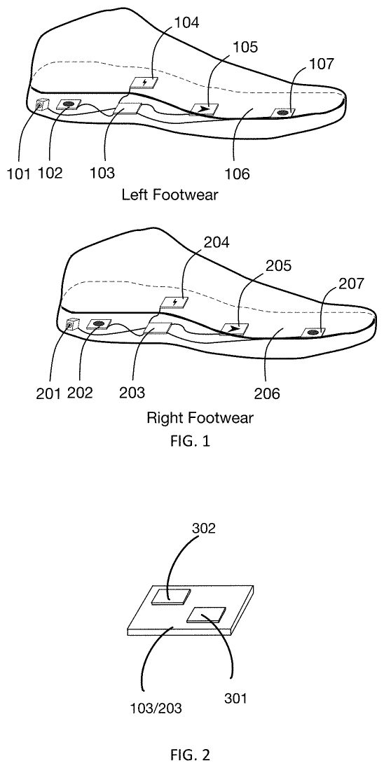

FIG. 1 illustrates components of an exemplary compass-sensor embedded footwear system in accordance with various embodiments of the present disclosure. The compass-sensor embedded footwear system illustrated in FIG. 1 may include a left footwear and/or a right footwear, which may include a shoe including a shoe body and a shoe sole. Although not shown in FIG. 1, all components in this exemplary system may be configured in a shoe insole.

In various embodiments, two pressure sensors 102 and 107 may be embedded in the left footwear sole 106 at locations corresponding to a bottom of a human left foot. For example, pressure sensor 107 may be positioned at a location corresponding to a center of a fore part (or a center of ball of foot) of a human left foot sole denoted as sole area A, pressure sensor 102 may be positioned at a location corresponding to a center of a heel part of a human left foot sole denoted as sole area B.

In various embodiments, two pressure sensors 202 and 207 may be embedded in the right footwear sole 206 at locations corresponding to a bottom of a human right foot. For example, pressure sensor 207 may be positioned at a location corresponding to a center of a fore part (or a center of ball of foot) of a human right foot sole denoted as sole area C, pressure sensor 202 may be positioned at a location corresponding to a center of a heel part (or a center of heel) of a human right foot sole denoted as sole area D.

In various embodiments, a compass sensor 105/205 may be embedded in the left/right footwear sole 106/206 or installed on the outer surface of the left/right footwear, at a fixed location and with a fixed orientation with respect to the left/right footwear sole 106/206. The compass sensor 105/205 is placed such that when the left/right footwear sole 106/206 is substantially leveled in a horizontal position, the compass sensor 105/205 is in normal operation.

The compass sensor 105/205 may be a 2-Axis digital compass. Alternatively, the compass sensor 105/205 may be a 3-Axis digital compass, especially when the compass sensor is tilted and not in a horizontally leveled position.

The compass sensor's operation relies on earth Magnetic Field. In weak earth magnetic field environments, compass sensors may not be able to produce accurate readings, which will affect performance of the compass-embedded footwear system. In such cases, as shown in FIG. 17 a strong artificial magnetic field can be created by placing one or more artificial magnetic source(s) 602 in close proximity of the compass-embedded footwear system when it is in user, so that the compass sensor may provide accurate directional measurements with respect to the artificial north reference direction.

In various embodiments, a control-communication unit 103/203 and a battery module 104/204 may be placed inside or on the outer surface of the left/right footwear 106/206 to support operation of the left/right footwear and its communication with e.g., external devices, such as smart phones, computers, game consoles, etc.

The control-communication unit 103/203, battery module 104/204, compass sensor 105/205 and pressure sensors 102,107/202,207 are connected with wires inside the left/right footwear for power, control and communication.

FIG. 2 further illustrates components of a control-communication unit 103/203, which has a processor module 301 and a wireless communication module 302, e.g., a blue tooth communication module.

For the compass-sensor embedded footwear system, various different configurations, placements, and/or arrangements of the battery module 104/204, control-communication unit 103/203, and compass sensor 105/205 may be included. This may in turn provide different tradeoffs among system performance, footwear appearance, and wearing comfort level.

In a first exemplary type of component arrangement configuration, or exemplary component arrangement configuration 1, the battery module 104/204, control-communication unit 103/203, and compass sensor 105/205, are all embedded in the footwear, for example in the footwear sole 106/206. In this configuration, a charging inlet 101/201 may also be provided on each footwear, either on a footwear sole or a footwear body. In some embodiments, the battery module may be wirelessly charged. In this case, the charging inlet 101/201 is optionally, and may or may not be configured.

The exemplary component arrangement configuration 1 in FIG. 1 has a minimum impact on appearance of the footwear. However, hiding all components inside the footwear bodies may negatively affect system performance in terms of operation hours per charge, and footwear wearing comfort level.

The exemplary component arrangement configuration 1 may allow the footwear to be in forms of shoes as shown in FIG. 1, although, not shown in FIG. 1, this exemplary component arrangement configuration 1 can be adapted into shoe insoles.

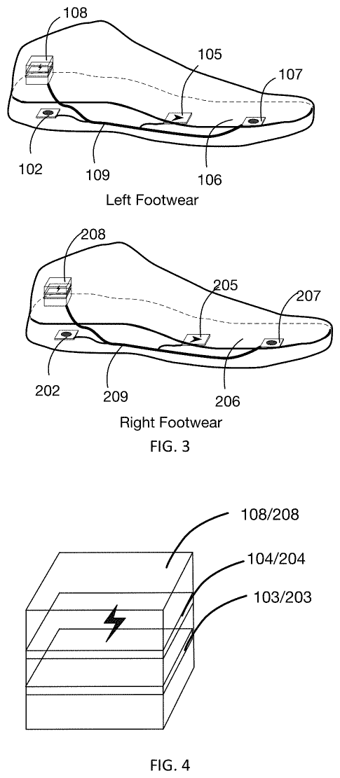

The second exemplary type of component arrangement configuration, or an exemplary component arrangement configuration 2, is illustrated in FIG. 3. In this configuration, the battery module and the control-communication unit are placed on the outer surface of the footwear, for example, attached to an outer surface of the footwear body and/or footwear sole of the footwear.

In various embodiments with component arrangement configuration 2, battery module 104/204 and the control-communication unit 103/203 may be combined as a single power-control-communication unit 108/208 as illustrated in FIG. 4. The single power-control-communication unit 108/208 may be installed on the outer surface of the footwear. The power-control-communication unit 108/208 is connected to sensors in the left/right footwear by cables 109/209 that run inside the left/right footwear for power supply, control and communication.

In various embodiments with component arrangement configuration 2, the power-control-communication unit 108/208 may be detachable and re-attachable to the footwear using a proper connector or clip.

Component arrangement configuration 2 may allow the separation of the left/right footwear into two parts. One part is a separate insole layer, containing the pressure sensors and the compass sensor. The other part is the power-control-communication unit. The two parts of the footwear may be connected by a proper connector and wires for power, control and communication. Note that the power-control-communication unit does not have any sensors in it and has the minimum installation requirement, which offers the most flexibility in its placement. It may be attached to the shoe body, ankle, lower leg, or any desired part of a user with proper fixtures, or it may be put into a pocket, as long as proper connections are made to the sensors.

Component arrangement configuration 2 may have certain impact on the footwear's appearance. However, installing components for power, communication and control outside the footwear may improve system performance, e.g., operation hours per charge, as well as footwear wearing comfort level. The optional detachable feature of the power-control-communication unit allowed by this component arrangement configuration may also make the charging, communication pairing and maintenance processes easier to improve user experiences, and support the use of the same left/right power-control-communication unit with different left/right footwear.

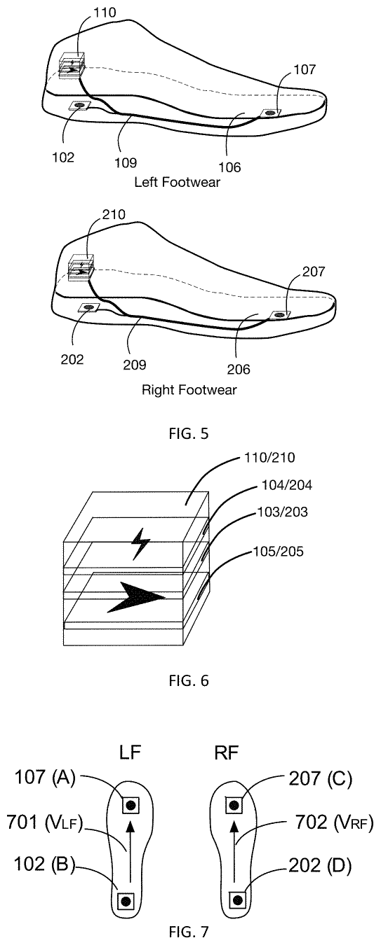

The third exemplary type of component arrangement configuration, or an exemplary component arrangement configuration 3, is illustrated in FIG. 5. In this configuration, the battery module, the control-communication unit and the compass sensor are placed on the outer surface of the footwear.

In various embodiments with component arrangement configuration 3, the battery module 104/204, control-communication unit 103/203, and the compass sensor 105/205 are combined into a single power-control-communication-compass unit 110/210. The power-control-communication-compass unit 110/210 illustrated in FIG. 6 may be installed on the outer surface of the footwear. The power-control-communication-compass unit 110/210 is connected to pressure sensors in the left/right footwear by cables 109/209 that run inside the left/right footwear for power supply, control and communication.

In various embodiments with component placement configuration 3, the power-control-communication-compass unit 110/210 may be detachable and re-attachable to the left/right footwear using a proper connector or clip. A detachable power-control-communication-compass unit 110/210 may also be used with different left/right footwear.

Component arrangement configuration 3 may allow the separation of the footwear into two parts. One part is a separate insole layer, containing only the pressure sensors. The other part is the power-control-communication-compass unit which is coupled with a shoe (e.g., including a shoe body and a shoe sole). The two parts of the footwear may be connected by a proper connector and wires for power, control and operation. Note that the power-control-communication-compass unit may be attached to a user's body part such as a user's ankle as long as the power-control-communication-compass unit has a fixed orientation with respect to the footwear sole when attached.

Compared to component arrangement configuration 2, component arrangement configuration 3 may further improve wearing comfort level of the compass-sensor embedded footwear system by leaving only the two pressure sensors inside the footwear sole. The footwear sole may be a separate insole layer or may be a shoe sole of the shoe footwear. However, it requires the power-control-communication-compass unit 110/210 to be placed such that it has a fixed orientation with respect to the footwear sole 106/206, and the integrated compass sensor 105/205 is in normal operation.

The disclosed system features a novel combined use of information from the left and right footwear. With the compass sensors, user's foot pointing directions, alternatively referred to as foot directional information, may be obtained in the user's local North-East coordinate system. The pressure sensors are able to provide pressure measurements at designed user sole areas. The foot directional information, used in conjunction with pressure measurements, may provide (user) directional information on a user's intended movements, and support complex foot gesture detections. The foot and/or user directional information and foot gesture detection results from the compass-sensor embedded footwear system may support various gaming applications and other types of applications for controls and, especially, hand-free navigation in simulated virtual world, to provide unique and improved user experiences.

For example, FIG. 7 illustrates arrangement of pressure sensors at four designed sole areas with respect to the contours of both human feet, and the corresponding left foot direction vector and right foot direction vector.

As shown in FIG. 7, the locations of the pressure sensors, e.g., on the designed sole areas A, B, C and D, may be with respect to contours of left and right soles. Sole area A corresponds to a center of a fore part (or a center of ball of foot) of a human left foot. Sole area B corresponds to a center of a heel part (or a center of heel) of a human left foot. Sole area C corresponds to a center of a fore (or a center of ball of foot) of a human right foot. Sole area D corresponds to a center of a heel part (or a center of heel) of a human right foot. At a certain data sampling time pressure level measurements from pressure sensors 102, 107, 202 and 207 may be denoted as PA, PB, PC and PC, respectively.

FIG. 7 also illustrates foot direction vectors V_{LF} (701) for a human left foot and V_{RF} (702) for a human right foot, which are aligned with the directions to which the corresponding foot is pointing at when a user is wearing the footwear.

FIG. 8 illustrates an exemplary relationship between the user's local North-East (N-E) coordinate system, a compass sensor's own reference X-Y 2D coordinate system, and a foot direction vector. In FIG. 8 the N axis corresponds to the user's local North direction, and the E axis corresponds to the user's local East direction. The Y axis corresponds to a compass sensor's reference 2D coordinate Y. The X axis corresponds to a compass sensor's reference 2D coordinate X. Angle θ_{L}/θ_{R} (705/706) is the angle from the North N axis to the Y axis, which can be obtained from compass sensor measurements.

Vector V_{LF} or V_{RF} corresponds to the foot direction vector 701/702 for left/right foot. Angle β_{L}/β_{R} (703/704) is the angle from the Y axis of a compass's reference coordinate system to a left/right foot direction vector 701/702. Once a compass sensor 105/205 is installed to the left/right footwear with a fixed orientation with respect to the left/right footwear sole 106/206, β_{L}/β_{R} (703/704) is fixed and can be easily measured/obtained. Angle co is the sum of 0 and 0, which is a foot (footwear) pointing direction angle in the user's local North-Each (N-E) coordinate system, i.e., the angle from the local North (N) axis to the foot direction vector. For left foot, the foot pointing direction angle ω is denoted as Ω_{L} (707), and for right foot, the foot pointing direction angle ω is denoted as Ω_{R} (708). For each foot, the local processor 301 is able to obtain θ (705/706) from the compass sensor and then evaluate the foot pointing direction angle ω (707/708) in the local North-East 2D coordinate system with the pre-obtained (703/704) of the corresponding left/right footwear.

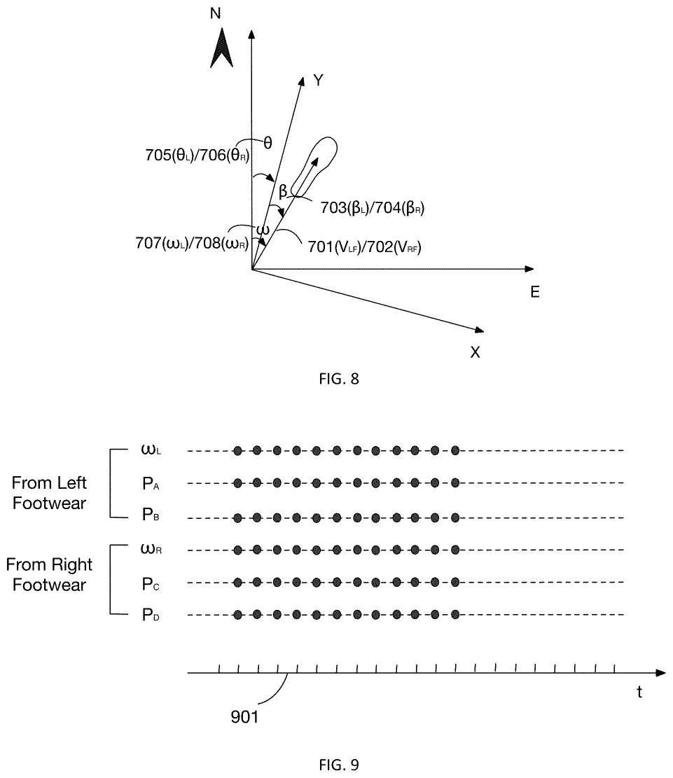

FIG. 9 summarizes the flows of measurements/derived measurements in the compass-sensor embedded footwear system over time. The sampling time interval is denoted as 901, which need not to be uniform. The uniform sampling time interval illustrated in FIG. 9 is only for illustration purpose. At each data sampling time, the compass sensor unit (105/205) of the left/right footwear provides a θL/θR measurement (705/706), which is used to obtain ωL/ωR (707/708) in the local N-E coordinate system with the pre-obtained β_{L}/β_{R} (703/704). The pressure sensors (107 and 102) of the left footwear provide pressure measurements PA and PB at the corresponding sole areas. The pressure sensors (207 and 202) of the right footwear provide pressure measurements P_{C} and P_{D} at the corresponding sole areas. At each sampling time, the obtained ω_{L} 707/ω_{R} 708, and pressure measurements (P_{A}, P_{B})/(P_{C}, P_{D}) form a left/right footwear measurement-information set.

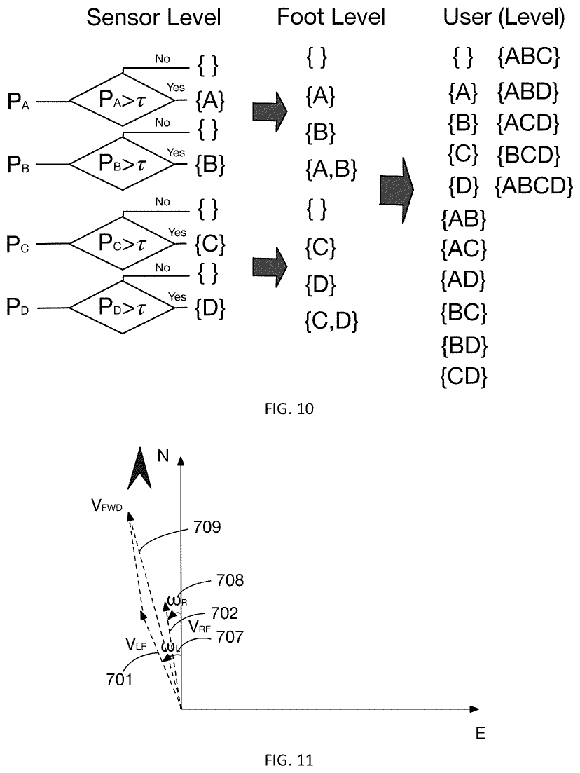

At a data sampling time, pressure measurements P_{A}, P_{B}, P_{C} and P_{D} may be used together to obtain a user (foot) touch detection outcome, i.e., the user foot touch state. One example of deriving user foot touch state based on pressure measurements P_{A}, P_{B}, P_{C} and P_{D} is illustrated in FIG. 10. Other methods can also be used to derive user foot touch state from pressure sensor measurements. As illustrated in FIG. 10, the pressure measurement from each sensor is first compared to a pre-set threshold level τ, above which a touch of the corresponding sole area touch is detected. The sensor-level sole area touch detection results that correspond to the left or right foot may be combined to produce foot-level touch detection results. As illustrated in FIG. 10, for each foot, a foot-level touch detection result, i.e., a single foot touch state, may fall in a set of four possible outcomes, denoted as { }, {A}, {B} and {A B} for left foot, and { }, {C}, {D}, {C D} for right foot. Combining the foot-level touch detection results for both feet, a user (level) touch detection outcome, i.e., a Bi-foot touch state, at a data sampling time may be obtained which has 16 possible outcomes, corresponding to 16 Bi-foot touch states, as the example listed in FIG. 10 ranging from { } (no-touch) to {A B C D} (full-touch). A user's single foot and Bi-foot touch state are basic/fundamental foot gesture features supporting the detection of various user foot gestures. In some embodiment of the disclosure, a user's foot touch state is determined by whether a fore part of a user's foot sole and a heel part of a user's foot sole touch/press a supporting surface/platform including the ground.

The foot pointing direction angles ω_{L} (707) and ω_{R} (708) obtained using measurements from the compass sensor unit (105/205) provide foot directional information or foot pointing directions in a common local N-E coordinate system, which may be further fused to obtain a user directional information. Note that compass sensor unit (105/205) may be a compass sensor, or be a compass sensor assembly including a compass sensor, and other sensors, e.g., accelerometer, which are physically combined as one platform. FIG. 11 illustrates fusion of ωL (707) and ωR to obtain a fused user (forward) directional vector V_{FWD} 709 that reveals the information on the wearer's (user's) intended movement direction. The fused user directional information can be provided as valuable user information/controls to other applications that run on external devices, e.g., a computer, a game console, and/or a smart phone, connected to the disclosed system through wireless communication links.

As shown in FIG. 11, foot pointing direction angles ω_{L} (707) and (OR (708) may be converted to foot direction vectors V_{LF} (701) and V_{RF} (702) in the local N-E coordinate system. FIG. 10 illustrates an exemplary simple way that may be used to obtain V_{FWD} (709) as the vector sum of V_{LF} (701) and V_{RF} (702). Other method for deriving V_{FWD} (709) may use the pressure measurements P_{A}, P_{B}, PC, and P_{D}, since, when a user applies different pressure to each foot, the pointing direction of the foot that has more pressure bears more information on V_{FWD}. For example, when a person stands on one foot, his or her natural movement direction is mostly determined by the pointing direction of the standing foot.

The derived user (forward) directional vector V_{FWD} 709 can also be used as a foot gesture feature to support the detection of new types of foot gestures.

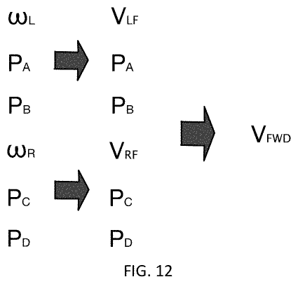

FIG. 12 illustrates an information processing flow to obtain V_{FWD} (709). First from the left and right footwear measurement-information sets, the foot pointing direction angles ω_{L} (707) and ω_{R} (708) are converted to foot direction vectors V_{LF} (701) and V_{RF} (702) in the local N-E coordinate system. Then the converted V_{LF} (701) and V_{RF} (702), along with pressure measurements P_{A}, P_{B}, P_{C}, and P_{D} may be processed jointly to produce the user (forward) directional vector V_{FWD}. For example, one processing method may be:

$${V}_{FWD} = \frac{{P}_{A} + {P}_{B}}{{P}_{A} + {P}_{B} + {P}_{C} + {P}_{D}} {V}_{LF} + \frac{{P}_{C} + {P}_{D}}{{P}_{A} + {P}_{B} + {P}_{C} + {P}_{D}} {V}_{RF}$$

which uses a weighted combination of V_{LF} and V_{RF} according to the pressure measurements. Another processing method to obtain V_{FWD} may be:

$${V}_{FWD} = \{ \begin{matrix}{V}_{LF} , & if {τ}_{1} < {P}_{A} + {P}_{B} and {P}_{C} + {P}_{D} \leq {τ}_{1} \\ \begin{matrix}\frac{{P}_{A} + {P}_{B}}{{P}_{A} + {P}_{B} + {P}_{C} + {P}_{D}} {V}_{LF} + \\ \frac{{P}_{C} + {P}_{D}}{{P}_{A} + {P}_{B} + {P}_{C} + {P}_{D}} {V}_{R F}\end{matrix} , & if {τ}_{1} < {P}_{A} + {P}_{B} and {τ}_{1} < {P}_{C} + {P}_{D} \\ {V}_{RF} , & if {τ}_{1} < {P}_{C} + {P}_{D} and {P}_{A} + {P}_{B} \leq {τ}_{1}\end{matrix}$$

where τ₁ is a pressure level threshold. If the total pressure level on a foot is below τ₁, the corresponding foot direction vector should not be used for the evaluation of V_{FWD}.

Other method for the evaluation of user directional information can be devised to best suit certain applications. In the present disclosure, the joint use of measurements from the pressure sensors at the designed sole areas, and the foot pointing directional information from the compass sensor unit (105/205) is able to provide valuable user (movement) directional information.

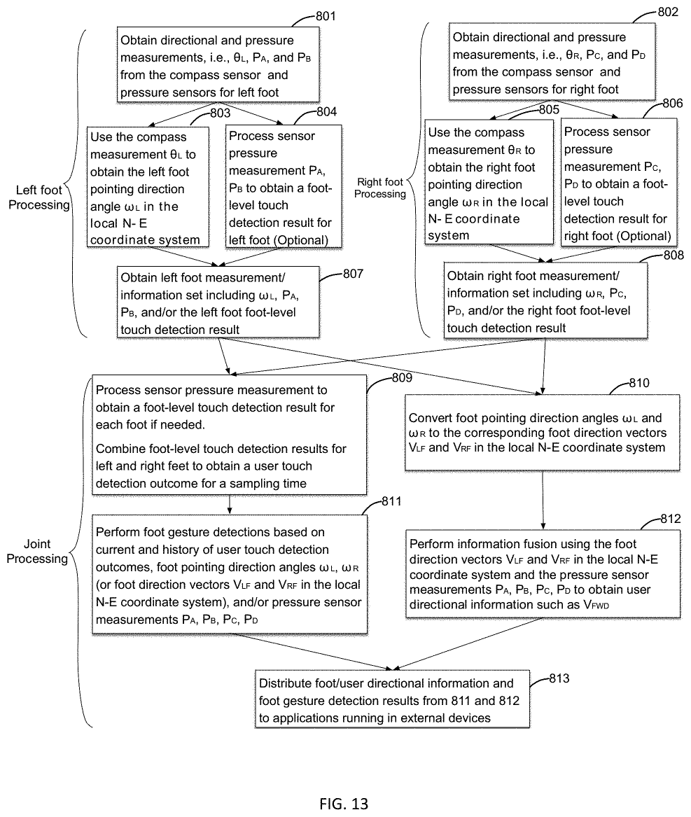

FIG. 13 summarizes the information processing flow at a data sampling time in an embodiment of the compass-sensor embedded footwear system. The processing flow starts from measurement collection at the left and the right footwear, as shown in Step 801 and Step 802, where compass measurement θ_{L} 705/0a 706 is read from the compass sensor unit 105/205 of the left/right footwear. In step 801/802, pressure measurements (P_{A}, P_{B})/(P_{C}, P_{D}) are read from the left/right footwear pressure sensors (102,107)/(202,207).

In Step 803/805, the compass measurements θ_{L}/θ_{R} (705/706) from Step 801/802 are processed with the pre-obtained β_{L}/β_{R} (703/704) to obtain ω_{L}/ω_{R} (707/708), which is the left/right foot pointing direction angle in the local North-East 2D coordinate system.

In Step 804/806, pressure measurements (P_{A}, P_{B})/(P_{C}, P_{D}) from Step 801/802 are processed according to FIG. 10 to obtain a foot-level touch detection result for the left/right foot. Note that this Step 804/806 is optional, since the same process can also be performed later in Step 809 as shown in FIG. 13.

In Step 807/808, results from Step 803/805 and 804/806 are combined to obtain a left/right footwear measurement-information set from the left/right footwear at each sampling time, including ω_{L}/ω_{R} (707/708) from step 803/805, (P_{A}, P_{B})/(P_{C}, P_{D}) and/or a left/right foot-level touch detection result from step 804/806.

By performing Steps 809 and 811, the measurement-information set from Step 807 for the left footwear and the measurement-information set from Step 808 for the right footwear are gathered together and jointly processed for foot gesture detections.

In Step 809, pressure measurements (P_{A}, P_{B}) from Step 807 for the left footwear and (P_{C}, P_{D}) from Step 808 for the right footwear are jointly processed to obtain the user touch detection outcome of the data sampling time, in the case that results from Step 807 and/or 808 do not have foot-level touch detection results. In the case that the left and right foot-level touch detection results are available from Steps 807 and 808, they can be directly combined in Step 809 to obtain the user touch detection outcome.

In Step 811, gesture detections are performed based on current and history of user touch detection outcomes from Step 809, foot pointing direction angles ω_{L}(707), ω_{R} (708) from Steps 807 and 808 (which may be converted to foot direction vectors V_{LF} (701) and V_{RF} (702) in the local N-E coordinate system), and/or pressure sensor measurements P_{A}, P_{B}, P_{C}, and P_{D} from Steps 807 and 808.

By preforming Steps 810 and 812, the measurement-information set from Step 807 for the left footwear and the measurement-information set from Step 808 for the right footwear are gathered together and jointly processed to obtain fused user directional information, such as V_{FWD} (709).

In Step 810, the foot pointing direction angles ω_{L} (707) and ω_{R} (708) from Steps 807 and 808 may be converted to the corresponding foot direction vectors V_{LF} (701) and V_{RF} (702) in the local N-E coordinate system.

In Step 812, the foot direction vectors V_{LF} (701) and V_{RF} (702) in the local N-E coordinate system from Steps 810 and the pressure sensor measurements P_{A}, P_{B}, P_{C}, and P_{D} from Steps 807 and 808 are further fused to obtain user directional information, such as V_{FWD}, according to various fusion methods devised for different types of applications.

In Step 813, foot gesture detection results from Step 811 and foot/user directional information from Step 812 are sent/dispatched to targeting applications of the compass-sensor embedded footwear system that may run in external (electronic) device(s).

Note that the processing flow for the compass-sensor embedded footwear system shown in FIG. 13 obtains information on the two basic types of foot gesture features, i.e., user foot pointing direction(s) and user foot touch state. The processing flow can be easily expanded to obtain information related to other additional types of foot gesture features. Also note that step 811 involves the detection of foot gestures, which can be done at either by the compass-sensor embedded system, or at an external electronic device with more processing power.

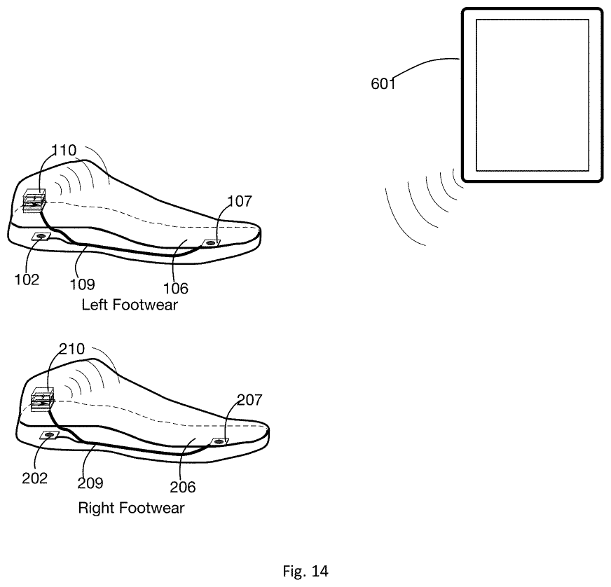

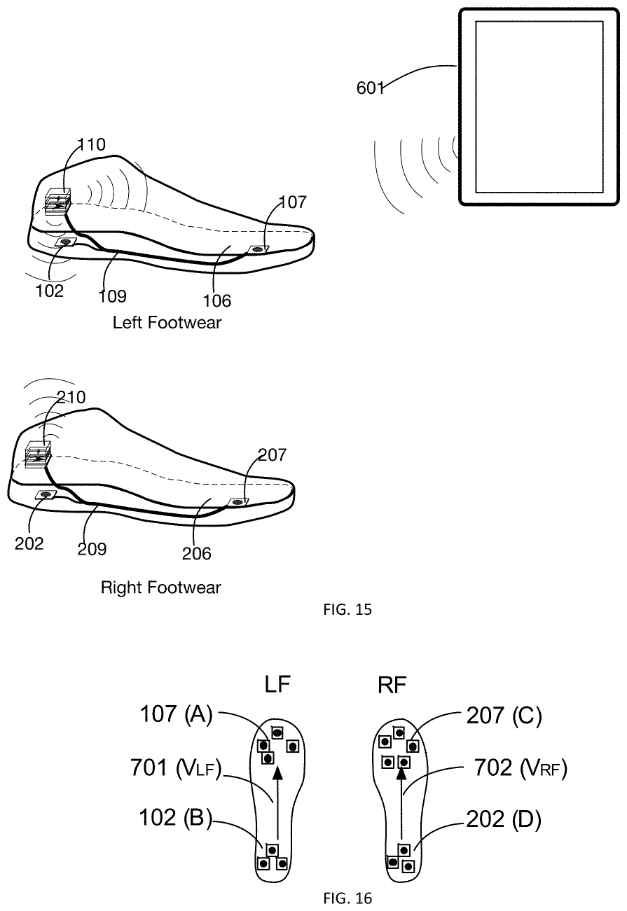

As such, by performing Steps 809, 810, 811 and 812, the information processing processes for foot gesture detections and user directional information extractions require the joint process of data and information from both feet. As a result, data and information originated from the left footwear and the right footwear need to be gathered together and processed at one place. Two types of system operation configurations may be used to address the problem, for example, as shown in FIGS. 14-15.

FIG. 14 illustrates an exemplary system operation configuration including a compass-sensor embedded footwear system and an external electronic device, where both the left footwear and right footwear communicate simultaneously with the external device

In the exemplary system operation configuration shown in FIG. 14, the joint processing of left and right foot information, e.g., Steps 809-812 in FIG. 13, is done at an external (electronic) device to which both the left footwear and the right footwear are connected. In this configuration, footwear measurement-information sets as shown in FIG. 9 and/or the foot-level touch detection results in FIG. 10 from both the left and the right footwear are sent through wireless links to the external device. The remaining processing for further fusion of the information and gesture detections are done at the external device by a software driver.

FIG. 15 illustrates another exemplary system operation configuration when the compass-sensor embedded footwear system works with an external device, where the footwear for one foot (e.g., left foot) is configured as master, and the footwear for the other foot (e.g., right foot) is configured as slave.

In the exemplary system operation configuration shown in FIG. 15, the joint processing of left and right foot information, e.g., Steps 809-812 in FIG. 13, is done at the left (or right) footwear that acts as a system master. The other footwear acts as a slave device. A wireless communication link, e.g., a blue tooth link, is established between the master and the slave. At the sampling times, the salve send its local measurement-information set, e.g., as illustrated in FIG. 9, and/or foot-level touch detection result, as illustrated in FIG. 10, to the master where the joint processing of left and right foot information may be performed. The master has another wireless communication connection with the remote device, through which controls/information requests for the sensor-embedded footwear system from the external device can be received, and foot directional information, user directional information and/or foot gesture detection results may be sent to the external device.

The different system operation configurations as illustrated in FIG. 14 and FIG. 15 show that the derivation of information on a foot gesture feature, e.g., the Bi-foot touch state can be done at a foot gesture feature information acquisition device such as the compass-sensor embedded footwear system (the case of FIG. 15), or at an electronic device using the foot gesture feature information (the case of FIG. 14).

Accordingly, a foot gesture feature information acquisition device obtains information related to foot gesture feature(s) and send the information to an electronic device. Information related to foot gesture feature(s) may be foot gesture features, i.e., information directly related to foot gesture features, or information needed to derive foot gesture feature information, i.e., information indirectly related to foot gesture features.

The present disclosure is able to obtain rich action and gesture feature information from human feet that are not available from existing hand operation based systems. Outputs from the present disclosure can be used for device control, video game applications, interactive 3D programs and virtual reality applications to support hand-free navigation in simulated virtual worlds.

The above detailed descriptions only illustrate certain exemplary embodiments of the present invention, and are not intended to limit the scope of the present invention. Those skilled in the art can understand the specification as whole and technical features in the various embodiments can be combined into other embodiments understandable to those persons of ordinary skill in the art. Any equivalent or modification thereof, without departing from the spirit and principle of the present invention, falls within the true scope of the present invention.

Note that all components of the disclosed compass-embedded footwear system illustrated in FIG. 1, FIG. 3, and FIG. 5 are for example only and only requires a basic set of sensors to achieve the disclosed functions of providing user/foot directional information, and foot gesture detections. Note that components in FIGS. 1, 3, and 5 may be omitted, modified, and/or altered, and more components may be added into the system exemplarily illustrated in FIGS. 1, 3, and 5. For example, any number of any types of components, including additional sensors, such as more pressure sensor(s), bend sensor(s), GPS sensor(s), heart rate sensor(s), gyroscope, accelerometer(s), haptic component(s), etc., may be additionally included in the system to add additional functions to the compass-embedded footwear system, or may be used to replace component(s) in the system. Each additional component(s) or sensor(s) mentioned above may be connected with the control-communication unit 103/203 for power, control and communication. For example, an additional component may be embedded in a footwear as shown in the component placement configuration 1 in FIG. 1, or the additional component (except for pressure sensors) may be integrated into the power-control-communication unit 108/208 as shown in component placement configuration 2 in FIG. 3, or the additional component (except for pressure sensors) may be integrated into the power-control-communication-compass unit 110/210 as shown in component placement configuration 3 in FIG. 5.

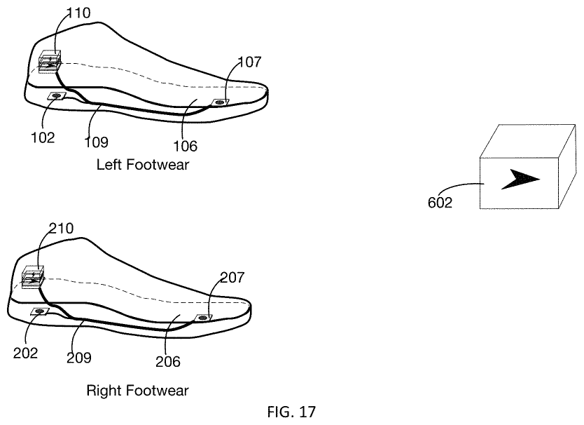

In an exemplary embodiment, each footwear, left or right footwear, may include two or more pressure sensors. FIG. 16 illustrates another exemplary footwear when each of the four pressure sensors in FIG. 1, FIG. 3, and FIG. 5 is replaced by a set of pressure or force sensors. In this case each pressure measurement, i.e., P_{A}, P_{B}, P_{C} or P_{D} can be derived based on readings from the corresponding set of pressure sensors. For example, P_{A}/P_{B}/P_{C}/P_{D} can be derived as the sum of the readings from the corresponding set of pressure sensors at the fore part of a user's left foot sole, left foot heel part, fore part of a user's right foot sole, and right foot heel part.

In addition to providing P_{A}, P_{B}, P_{C} or P_{D} measurement, the pressure sensor sets as illustrated in FIG. 16, can also provide detailed pressure distribution information over the user's feet. The pressure distribution information can be used to support more sophisticated foot gesture detections. For example, the pressure distribution information can be used to differentiate whether a user's foot is pressing the ground using inner side of a left/right footwear or using outer side of a left/right footwear, or the whole left/right footwear.

In addition to the two sets of pressure sensors, more pressure sensors may be available for the rest of the left/right foot areas. These sensors form a third set of pressure sensors, which, together with the previous two pressure sensor sets, provides detailed pressure distribution information over the entire left/right sole. The additional sole pressure distribution information can be used to support the detection of new types of foot gestures.

Based on the discussion above, there may be multiple, e.g., two and two plus, pressure/force sensors in the left/right footwear.

For example, bend sensors, such as resistive bend sensors, can be added to the compass embedded footwear system to provide additional information on the bending status of the foot sole(s) for the detection of new types of foot gestures. The resistive bend sensor(s) can be connected with the control-communication unit.

Motion sensors including gyroscope (angle rate) sensors, accelerometers and combined gyroscope-accelerometers, may be added to the compass embedded footwear system to provide information such as foot acceleration information and angular velocity information. Note that, while foot motions may be detected by indirectly comparing compass measurements at different times, a compass sensor is distinguished from a motion sensor, since the compass sensor provides only directional information. The motion sensor used herein may refer to individual sensors, such as gyroscope and/or accelerometer, as well as any combination of multiple types of sensors used for motion detection. The motion sensors may be place inside or outside of the footwear and properly connected through wires for power, control and communication. Like the compass sensor, the motion sensor(s) are normally arranged to have a fixed orientation with respect to the foot sole. As a result, the motion sensor(s) have a fixed orientation with respect to the compass sensor. Information from the motion sensor, such as gyroscope sensors and/or accelerometers, is able to detect and characterize the user's foot movement, and can be used as new foot gesture features or used to derive other foot gesture feature information to support new types of foot gesture detections along with the foot directional information from the compass sensor. For example, for foot gesture detections, information from motion sensors, such as gyroscope sensors and accelerometers, can be jointly used with the foot directional information from the compass sensor to support the detection of fast and/or slow kicking movements in various directions. As another example, accelerometers are often used to support 3-axis compass sensors, where foot roll and pitch angle measurements from an accelerometer can be jointly used with measurements from a 3-axis compass sensor for tilt compensation. A tilt compensated compass sensor is able to give more accurate readings when the compass operates in a titled position.

In fact, accelerometer and gyro sensors are important to the compass-sensor embedded footwear to provide stable and accurate foot pointing direction information, as well as the accurate detection of sudden changes in foot pointing direction. This is because positions of the compass sensor unit 105/205 will not remain at a position that is level to the ground due to user's foot position changes and various foot movements. In a titled (unleveled) position, a 2-axis or 3-axis compass sensor alone cannot give accurate direction information because the magnetic field projects differently on the sensor's axes in a tilted position. Information for a 3-axis accelerometer need to be used for tilt compensation to recover the compass sensor measurements that would have been obtained by the compass sensor in a position leveled to the ground.

On the other hand, tilt compensation with 3-axis accelerometer measurements is only effective when the sensors are relatively stationary such that accelerometer measurements in x, y and z axes can be used to derive tilt information (roll and pitch angles) of the sensor platform accurately. For the derivation of foot pointing direction angle in presence of significant user foot movement, such as foot wiggle movement, measurements from a 3-axis gyro sensor may be effectively used. The gyro sensor provides angle rate information of the sensor platform in 3 axes. Depending on the sensor position relative to user's foot, angle rate measurements in one of the x, y and z axes or a certain combination of the gyro measurements in all 3 axes may be effectively used as turn rate of the user foot pointing direction. With the foot pointing direction turn rate, changes in foot pointing direction can be effectively derived by integration of the turn rate over time; and the foot pointing directions can also be derived based on the derived direction changes.

In summary when a user's foot has no significant movement a 3-axis compass sensor and a 3-axis accelerometer are used together as a tilt compensated compass to provide accurate foot pointing direction information in different foot positions; when a user's foot has significant movement, angle rate measurements from a 3-axis are used to derive changes in the foot pointing direction; the detection of significant user foot movement can be easily achieved using measurements from the 3-axis accelerometer and/or the 3-axis gyro sensor. As a result, for desired system performance in deriving accurate foot pointing direction information in various practical conditions, in embodiments of the invention the compass sensor unit 105/205 is a 9-axis senor unit including a 3-axis compass, a 3-axis accelerometer and a 3-axis gyro sensor.

Note that with tilt compensation, the compass sensor unit 105/205 can in principle operates in any orientations. As a result, the compass sensor unit 105/205 does not need to be installed in a leveled position, which offers more freedom in sensor placement. However, the orientation of the compass sensor unit 105/205 still need to have a fixed orientation w.r.t the user's foot.

Besides foot pointing direction information, and foot touch state, when the compass sensor unit 105/205 is a 9-axis sensor unit, including a 3-axis compass sensor, a 3-axis accelerometer, and a 3-axis angle rate (gyro) sensor, information on additional foot gesture features can be obtained by the compass-sensor embedded system. These foot gesture features include foot tilt angle(s), foot roll angle(s) and various foot moving trajectory state related foot gesture features, which will be detailed as follows.

As discussed earlier, in the compassed embedded footwear system, foot tilt angles can be derived using measurements from the 3-axis accelerometer in the compass sensor unit 105/205 when a user foot is not in significant movement. When a user foot is in significant movement changes in foot tilt angle can be effectively tracked using measurements from a 3-axis gyro sensor in the 9-axis compass sensor unit.

For example, assume, without loss of generality, the compass sensor unit 105/205 is installed in a position such that the plane formed by the x-axis and y-axis of the gyro sensor is parallel to the user foot sole surface, and the y-axis is the same as the left/right 3D foot pointing direction V_{LF}3D/V_{RF}3D (1003/1004). Assuming the foot roll angle λ_{L}/λ_{R} (1005/1006) is small and negligible. As illustrated in FIG. 29, the variation/change in foot tilt angle γ_{L}/γ_{R} (1001/1002) corresponds to the rotation around the gyro sensor's x-axis, i.e. Xgyro, which can be derived by integrating the x-axis angle rate measurements from the gyro sensor over time. In other sensor placement configuration, variation/change in foot tilt angle γ_{L}/γ_{R} (1001/1002) can also be similarly derived using gyro sensor measurements.

As described earlier, to offer desired foot pointing direction measure performance, the compass sensor unit 105/205 is a 9-axis senor unit including a 3-axis compass, a 3-axis accelerometer and a 3-axis gyro sensor. In such a configuration, measurements from the accelerometer can be used to derive the foot tilt angle information.

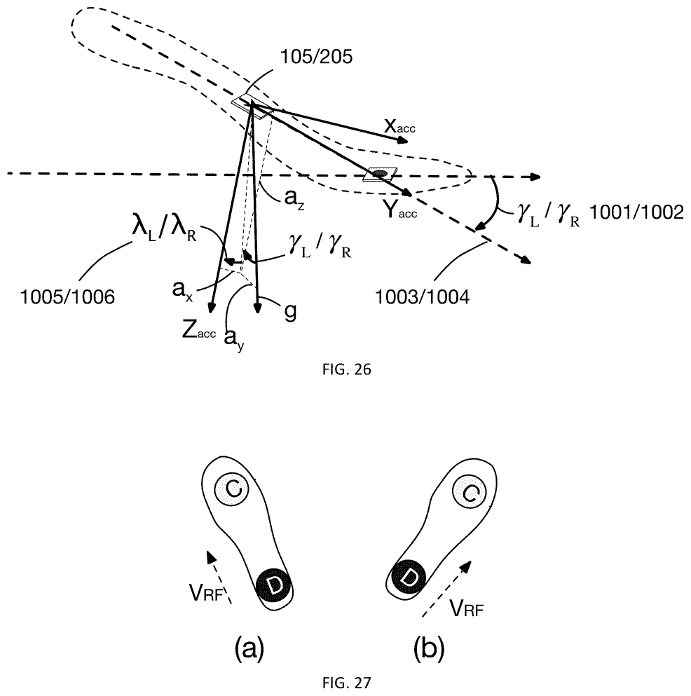

For example, assume, without loss of generality, the compass sensor unit 105/205 is installed in a position such that the plane formed by the x-axis and y-axis of the accelerometer is parallel to the user foot sole surface, and the y-axis is the same as the middle line of the left/right foot sole (1003/1004) (as a result the foot pointing direction) as shown in FIG. 26. In such a sensor place configuration, the foot tilt angle γ_{L}/γ_{R} of the left/right foot, i.e., γ_{L}/γ_{R} ((1001/1002) can be obtained as

$$γ = a tan \frac{{a}_{y}}{\sqrt{{a}_{x}^{2} + {a}_{z}^{2}}} ,$$

where aₓ, a_{y} and a_{z} are measurements in x, y, and z axes (denoted as x_{acc}, y_{acc}, and z_{acc} in FIG. 26) of the accelerometer in 105/205. As illustrated in FIG. 26 measures aₓ, a_{y} and a_{z} are projections of the gravity vector g to the accelerometer's x, y and z coordinates. In other sensor placement configurations, the foot tilt angle can be derived from the 3-axis accelerometer measurements with proper linear coordinate conversions.

As shown in FIG. 26, another angle in the x-y plane of the accelerometer's coordinate system λ_{L}/λ_{R} (1005/1006) corresponds to the roll angle of a user's left/right foot. This angle can also be derived using measurements from the accelerometer in the compass sensor unit (105/205) and be used as additional foot gesture parameters when needed.

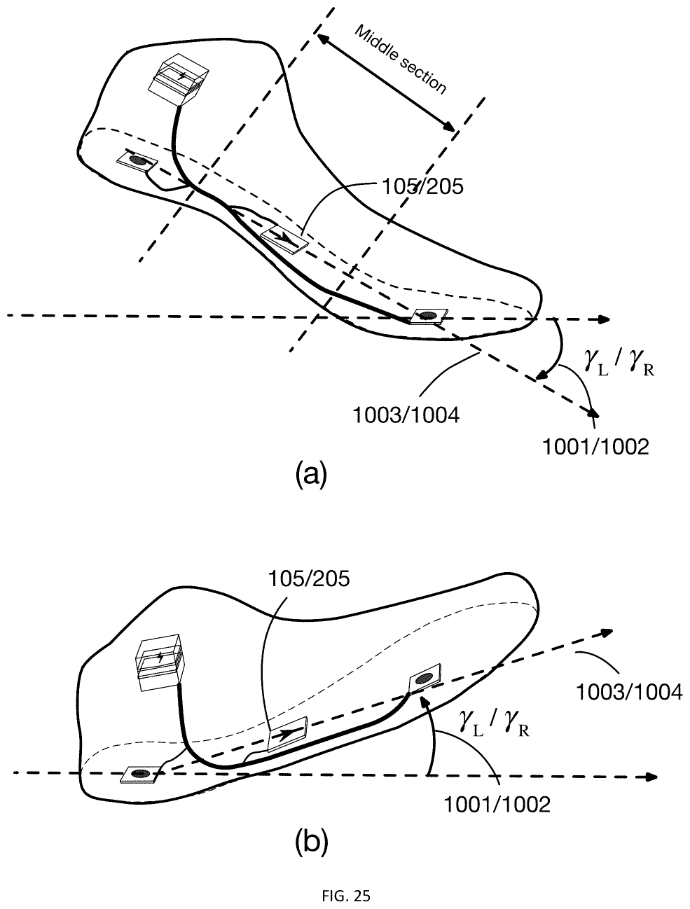

Note that to obtain desired foot tilt angle measurements for both cases with fore part of a user's foot sole touching (pressing) the ground (as in FIG. 25a) and with heel part of a user's foot sole touching (pressing) the ground (as in FIG. 25b), the compass sensor unit 105/205 should be placed close to the middle section (in length direction) of the user foot or foot sole as illustrated in FIG. 25.

Also note that the use of 3-axis accelerometer measurements to obtain foot tilt angles requires that the user's foot is not having significant movements.

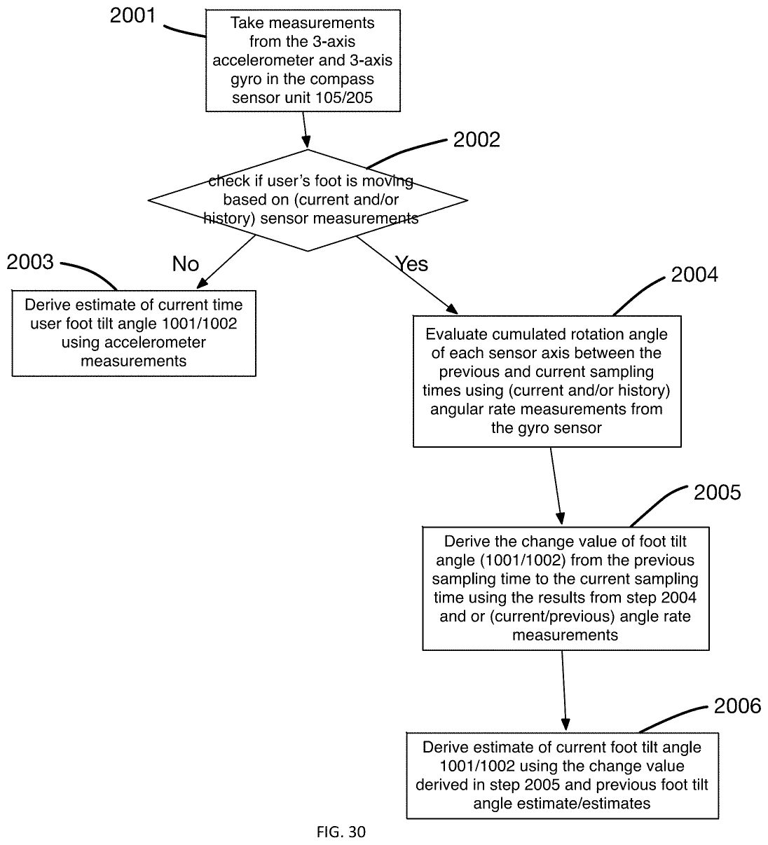

For the derivation (estimation) of foot tilt angle γ_{L}/γ_{R} (1001/1002) when a user's foot is moving or stationary the processing flow in FIG. 30 can be used. To achieve desired estimation accuracy, the processing flow in FIG. 30 should be executed at a sample rate that is sufficiently high. At each sampling time, the processing starts from step 2001 by taking measurements from the 3-axis accelerometer and gyro sensor in the 9-axis compass sensor unit 105/205. Using the sensor measurements obtained from step 2001, a check is performed at step 2002 to decided whether the foot is currently in significant movement. Such a check can be done using 3-axis accelerometer meter measurements to check if the total acceleration value matches the gravity value, e.g. around 9.8 m/s², or using 3-axis gyro sensor measurements to see if there is any significant angle rate measured in each gyro sensor axis, or the combination of both. If the check in step 2002 decides that the user foot is not moving, an estimate of current user foot tilt angle 1001/1002 is derived in step 2003 using 3-axis accelerometer measurements (as discussed early). If the check in step 2002 decides that the user foot is moving, step 2004 evaluates the rotated angle of each sensor axis between the previous and current sampling times using (current and/or history) angular rate measurements from the gyro sensor. Then, using the results from step 2004 and or (current/previous) angle rate measurements, step 2005 derives the change (increased or decreased) value of foot tilt angle (1001/1002) from the previous sampling time to the current sampling time. Then at step 2006, estimate of current foot tilt angle 1001/1002 is derived using the foot tilt angle change value from step 2005 and previous foot tilt angle estimate/estimates (in case any low pass filtering should be performed).

Similarly, the derivation of user foot pointing direction (i.e., the vector V_{LF}/V_{RF}) (in 2D local X-Y plane) using compass measurements with tilt compensation (using the accelerometer measurements) also requires the user foot to be roughly stationary. When a user foot is in significant movement changes in foot pointing direction can also be effectively tracked using measurements from a 3-axis gyro sensor in the 9-axis compass sensor unit.

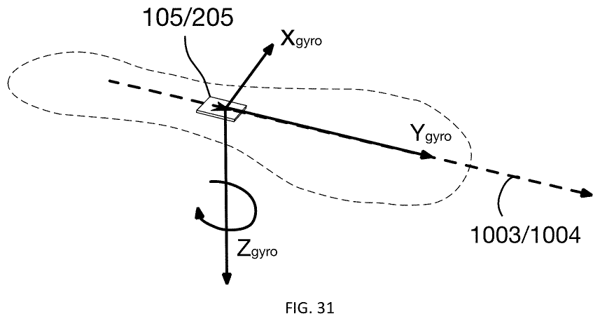

Again, assume the compass sensor unit 105/205 is installed in a position such that the plane formed by the x-axis and y-axis of the gyro sensor is parallel to the user foot sole surface, and the y-axis is the same as the left/right 3D foot pointing direction V_{LF}3D/V_{LF}3D (1003/1004). As illustrated in FIG. 31 shows, the foot pointing direction changes as the foot rotate around the gyro sensor's z-axis. When the user's foot is in a roughly leveled position, i.e., with small foot roll angle λ_{L}/λ_{R} (1005/1006) and tilt angle γ_{L}/γ_{R} (1001/1002), changes in user 3D foot pointing direction (1003/1004) can be well estimated by the angle rotated around the gyro sensor's z-axis. As a result, with angular rate measurements from z-axis of the gyro sensor, the changes in foot pointing direction (increased or decreased amount in foot pointing angle ω_{L}/ω_{R} (707/708), or changes in foot pointing direction vector 701/702) over a time period can be obtained. For cases with other sensor placement configuration, and with significant foot tilt and roll angles the changes in foot pointing direction can be similarly derived with proper coordinate transform and mapping.

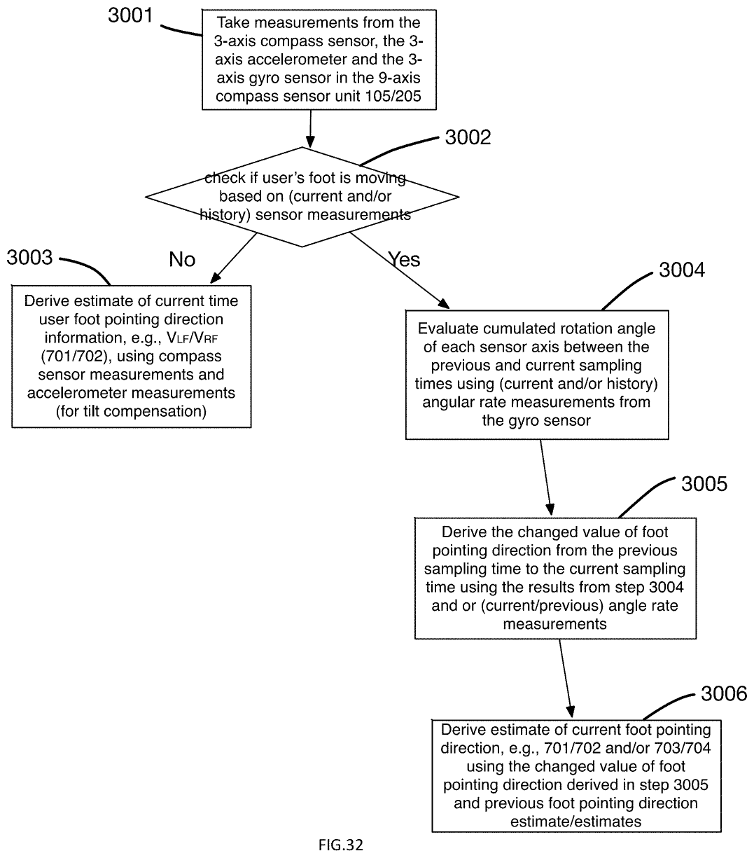

For the derivation (estimation) of foot pointing direction, e.g., foot pointing direction vector V_{LF}/V_{RF} (701/702) or foot pointing angle ω_{L}/ω_{R} (707/708), when a user's foot is moving or stationary the processing flow in FIG. 32 can be used. To achieve desired estimation accuracy, the processing flow in FIG. 32 should be executed at a sample rate that is sufficiently high. At each sampling time, the processing starts from step 3001 by taking measurements from the 3-axis compass sensor, the 3-axis accelerometer and the 3-axis gyro sensor in the 9-axis compass sensor unit 105/205. Using the sensor measurements obtained from step 3001, the same check as in 2002 is performed at step 3002 to decide whether the foot is currently in significant movement. If the check in step 3002 decides that the user foot is not moving, an estimate of current user foot pointing direction in the form of a foot pointing direction V_{LF}/V_{RF} (701/702) or foot pointing angle ω_{L}/ω_{R} (707/708) is derived in step 3003 using measurements from the 3-axis compass sensor and 3-axis accelerometer measurements (for tilt compensation). If the check in step 3002 decides that the user foot is moving, step 3004 evaluates the rotated angle of each sensor axis between the previous and current sampling times using (current and/or history) angular rate measurements from the gyro sensor. Then, using the results from step 3004 and or (current/previous) angle rate measurements, step 3005 derives the change (increased or decreased) value of foot pointing direction angle ω_{L}/ω_{R} (707/708) and/or the foot pointing direction vector V_{LF}/V_{RF} (701/702) from the previous sampling time to the current sampling time. Then at step 3006, estimate of current foot pointing direction in the form of a foot pointing direction V_{LF}/V_{RF} (701/702) and/or foot pointing angle ω_{L}/ω_{R} (707/708) is derived using the foot pointing direction change value from step 3005 and previous foot pointing direction estimate/estimates (in case any low pass filtering should be performed).

The follows describe how to obtain foot moving trajectory state estimates/information using measurements from some embodiments of the compass embedded footwear system. The obtained foot moving trajectory states can be used as foot gesture features and derive a range of foot gesture features related to user's foot moving trajectories.

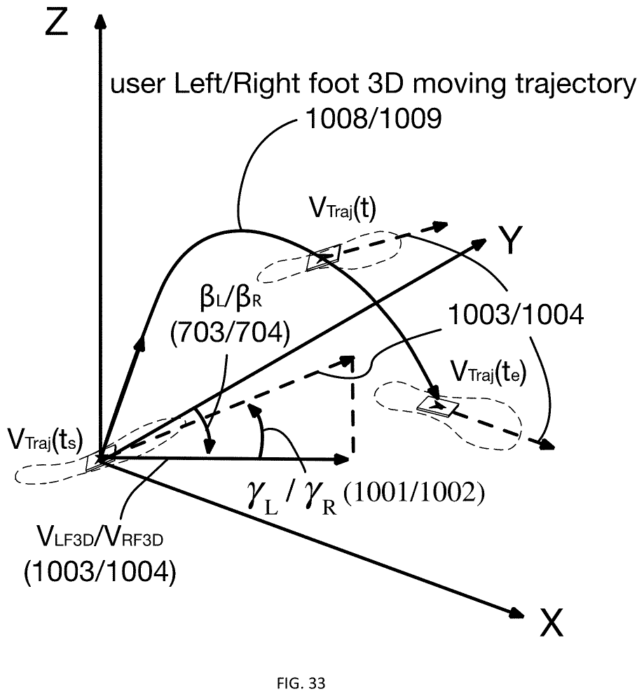

With the 9-axis compass sensor unit (105/205), the trajectory of a user's foot or equivalently the trajectory of the center point of the compass sensor unit (105/205) in the fixed/non-rotating 3D coordinate system (that does not change or rotate with user movements) can be obtained. As before, it is assumed that the X-Y plane of the fixed/non-rotating coordinate system is leveled or parallel to the local North-East coordinate plane. The estimation of user foot moving trajectory involves the estimation of a trajectory states from a starting (time) point is to an end (time) point te. As illustrated in FIG. 33, it can be assumed that the origin of the fixed/non-rotating 3D coordinate system, i.e. coordinate (0,0,0) is at the starting point of the foot moving trajectory (1008/1009). The trajectory state can be defined as a 9 dimensional state vector V_{Traj}=[x, y, z, vx, vy, vz, ax, ay, az]′, where x, y, and z are coordinates of the trajectory in the fixed 3D coordinate system, vx, vy and vz are velocities components of the trajectory state vector V_{Traj}, ax, ay and az are acceleration components of V_{Traj}. The full user foot moving trajectory (1008/1009) is a continuous function of the state vector between the time point, i.e.,

V_{Traj}(t)=[x(t),y(t),z(t),vx(t),vy(t),vz(t),ax(t),ay(t)az(t)]′,t∈[ts te].

In practice trajectory state VTraj is not estimated in continuous time by estimated in discrete time. Assuming a sampling time interval T, the trajectory state can be estimated at every sampling time, i.e. 0, T, 2T, 3T, . . . kT, . . . KT (assuming 0 corresponds to the starting time ts and KT corresponds to the end time te. Then the following state transition equation is satisfied

V_{Traj}[(k+1)T]=A(T)*V_{Traj}[kT],k=0,1,2,3, . . . K−1

where V_{Traj}[kT] denotes the trajectory state at discrete time, i.e.,

V_{Traj}[kT]=[x[kT], y[kT], z[kT], vx[kT], vy[0], vz[kT], ax[kT], ay[kT] az[kT]]′

and A(T) is a constant linear state transition matrix of dimension 9 by 9 for given sampling time T.

For the estimation problem, at each sampling time T, measurements of the acceleration state components ax(t), ay(t), az(t) at sampling time kT, denoted as ax[kT], ay[kT] and az[kT] can be obtained using measurements from the compass sensor unit (105/205). Denote the vector of acceleration trajectory state components A_{Traj}[kT]=[ax[kT], ay[kT], az[kT]]′. One has the following linear observation model for the trajectory state at a sampling time A_{Traj}[kT]=H*V_{Traj}[kT], where H is a 3 by 9 observation matrix

$$H = [ \begin{matrix}0 & 0 & 0 & 0 & 0 & 0 & 1 & 0 & 0 \\ 0 & 0 & 0 & 0 & 0 & 0 & 0 & 1 & 0 \\ 0 & 0 & 0 & 0 & 0 & 0 & 0 & 0 & 1\end{matrix} ]$$

Note that measurement of the acceleration component az[kT] should be obtained by subtracting the gravity acceleration g from the converted acceleration measurement in the Z axis of the fixed 3D coordinate system.

With the formulation above, the trajectory state V_{Traj}[kT] can be estimated over time using the sampled acceleration measurements A_{Traj}[kT] when the initial state at the starting time point k=0, i.e., V_{Traj}[0] is available. Note that the foot moving trajectory estimation problem formulated above is a typical linear dynamic state estimation problem. Detailed state transition model and measurement model are not discussed here.

The initial trajectory state V_{Traj}[0] at the trajectory starting time to is in general easy to obtain. Since it is assumed earlier that the trajectory starts from the origin of the coordinate system, one has V_{Traj}[0]=[0, 0, 0, vx[0], vy[0], vz[0], ax[0], ay[0] az[0]]′.

It is convenient to set the starting point of the foot moving trajectory (1008/1009) when the user foot is not moving and thus has zero velocity components. As a result, the initial trajectory state vector is given by V_{Traj}[0]=[0, 0, 0, 0, 0, 0, ax[0], ay[0], az[0]]′.

The missing components of the initial trajectory state vector are the acceleration components ax[0], ay[0] and az[0], which can be obtained from sensor measurements from the 9-axis compass sensor unit.

The only remaining challenge for foot moving trajectory state estimation is to obtain measurements of user foot acceleration (or equivalently compass sensor unit (105/205) acceleration) in the fixed 3D coordinate system, i.e., A_{Traj}[kT]=[ax[kT], ay[kT], az[kT]]′. However, sensor measurements from the 3-axis accelerometer are obtained in the sensor's coordinate system along axis Xacc, Yacc, and Zacc as shown in FIG. 26. The accelerometer measurements need to be converted to the fixed 3D coordinate system. With foot pointing (heading) angle ω_{L}/ω_{R} (707/708), foot tilt angle γ_{L}/γ_{R} (1001/1002), and foot roll angle λ_{L}/λ_{R} (1005/1006), which are equivalent to the heading, tilt and roll angles of the compass sensor unit (105/205), acceleration measurements in the accelerometer's coordinate system can be mapped (converted) to the local fixed 3-D coordinate system using linear transformation.

The estimation of tilt angle γ_{L}/γ_{R} (1001/1002) and foot pointing (heading) angle ω_{L}/ω_{R} (707/708) using measurements from the 9-axis compass sensor unit has been presented previously. The estimation of foot roll angle λ_{L}/λ_{R} (1005/1006) using the 9-axis sensor measurements can be done using a method that is similar for the estimation of the tilt angle γL/γR (1001/1002). As a result, tilt angle γ_{L}/γ_{R} (1001/1002), foot pointing (heading) angle ω_{L}/ω_{R} (707/708) and foot roll angle λ_{L}/λ_{R} (1005/1006) can all be estimated using measurements from a 9-axis compass sensor unit (105/205).

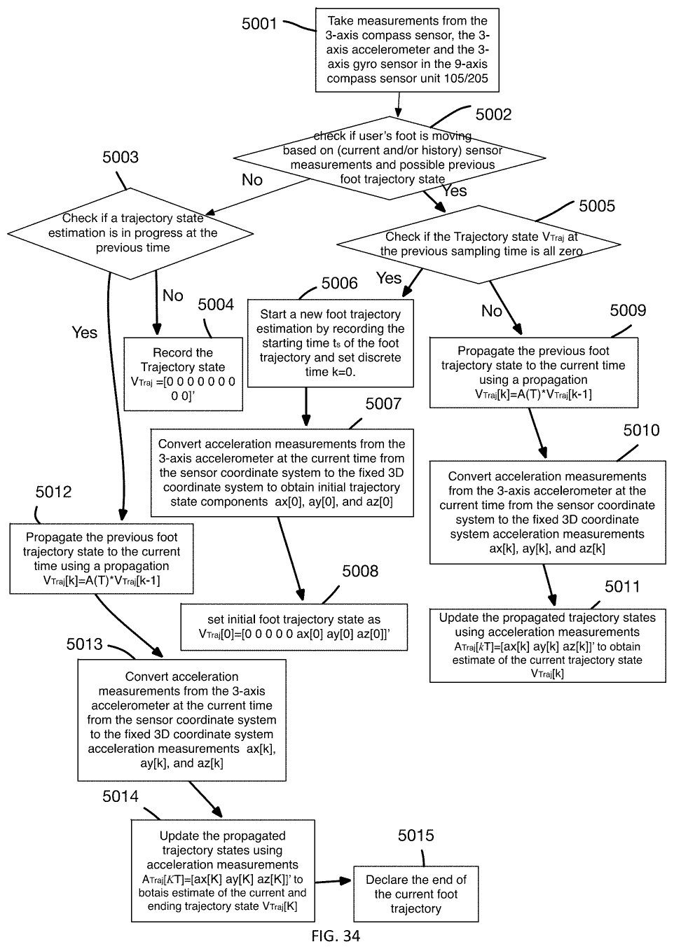

Based on the discussion above, the estimation of foot moving trajectory can be done using the processing flow shown in FIG. 34. It is assumed that the estimation process in FIG. 34 is conducted every sampling period T. At each sampling time, the foot moving trajectory state estimation process starts from step 5001 by taking measurements needed for the 9-axis compass sensor unit (105/205). Then at step 5002 a similar check as in 2002 is performed to decided whether the foot is currently in movement based on the current sensor measurements and possible foot moving trajectory state from previous sampling time. If no foot movement is detected at step 5002, step 5003 decide if there is an ongoing foot moving trajectory estimation process from the previous sampling time. If there is an existing foot moving trajectory estimation, step 5012 to step 5015 update the foot moving trajectory state V_{Traj}(K) at the current step and conclude (terminate) the current trajectory state estimation. Back to step 5003, if the check finds that there is no ongoing current trajectory state estimation, step 5004 simply set the current foot moving trajectory state as all zero elements and finish the processing at the current sampling step. Back to step 5002, if the check at step 5002 decides that the user's foot is in motion, step 5005 check if there is an ongoing foot moving trajectory state estimation based on the results from the previous sampling time, e.g., all-zero foot moving trajectory state V_{Traj} indicates there is no ongoing foot moving trajectory estimation. If all-zero foot moving trajectory state is found at the previous sampling time (corresponding to no ongoing foot moving trajectory estimation), steps 5006, 5007 and 5008 perform a foot moving trajectory state estimation initialization process to start a new trajectory estimation and obtain the initial state of the foot moving trajectory, i.e., V_{Traj} [0]. Note that in step 5013, the conversion of acceleration measurements from the accelerometer to the fixed 3D coordinate system needs foot tilt angle γ_{L}/γ_{R} (1001/1002), foot pointing (heading) angle ω_{L}/ω_{R} (707/708) and foot roll angle λ_{L}/λ_{R} (1005/1006) at the sampling time, which are available from supporting estimation processes using previously discussed estimation methods. Back to step 5005, if the check finds that the foot moving trajectory state vector V_{Traj} at the previous sampling time has non-zero elements, i.e., there is an ongoing foot moving trajectory state estimation, steps 5009, 5010 and 5011 conduct foot moving trajectory state estimation at the current sampling time. First in step 5009, the foot moving trajectory state at the previous sampling time will be propagated to the current sampling time. Then in step 5010, acceleration measurements in the fixed 3D coordinates at the current time are obtained using the same approach as in step 5007. In step 5011, the updated foot moving trajectory state V_{Traj}[k] at the current sampling step is obtained by updating the propagated foot moving trajectory state from step 5009 with the acceleration measurements obtained in step 5010. The state estimation process in steps 5009, 5010 and 5011 are the same as those in steps 5012, 5013 and 5014.

In general, a foot moving trajectory can be associated with a foot gesture using time matching. For example, assuming a foot moving trajectory is detected with a length of K sampling times with a trajectory state sequence from V_{Traj}[0], V_{Traj}[1], V_{Traj}[2] V_{Traj}[K]. If the starting time of a foot gesture (or a section of a foot gesture) corresponds to the sampling time of one of the trajectory state k1∈0, 1, . . . , K, and the ending time of a foot gesture (or foot gesture section) corresponds to another sampling time of the trajectory state k2∈0, 1, . . . , K, and k1<k2, then foot gesture (foot gesture section) can be associated with the 3D foot moving trajectory corresponding to trajectory states ranging from V_{Traj}[k1] to V_{Traj}[k2].

Other types of sensors can be incorporated into the compass-sensor embedded system which is discussed as follows.

GPS sensor or sensors may be added to the compass-embedded footwear system to provide information on foot/user location and velocity. In this case the GPS sensor may be located inside or outside of the left/right footwear and properly connected with the control-communication unit through wires for power, control and communication. Location, velocity and acceleration measurements from the GPS sensor can be gathered by the processor 301, processed, used, and/or distributed through the wireless connection to external devices. Information from the GPS sensor may be used to support the detection of new types of foot gestures. User location information from the GPS sensor may also be used to derive the local declination information, which accounts for the angle between the local earth's magnetic field and the physical North direction, and allows the compass to produce more accurate North direction measurements.

Haptic types of components can be added to the compass-embedded footwear system to provide vibration feedback to the user's feet. In such cases, a haptic component may be placed inside the footwear beneath the user's left/right sole. The haptic component should be properly connected for power, control and communication, e.g., be connected with the control-communication unit. The haptic component is able to give user vibration feedbacks that can be jointly used with the existing functions of the compass-embedded footwear system. For example, when a user is currently using a map application with a mobile device such as an iPhone for navigation during a walk, the application cannot tell which direction the user is about to move to, unless the user moves toward that direction for some distance. With the user directional information from the disclosed system such as V_{FWD} 709, the map application will be able to tell the direction to which the user is about to move even when the user is standing still. Note that other types of sensors may rely on the user's movement to obtain directional information. As a result, this feature can only be provided by the disclosed compass-embedded footwear system. Based on the information, the application may inform the user if the user is moving or about to move toward the correct direction that follows a route by sending vibration feedbacks through the haptic component/components in the footwear. The application can even guide the user to turn left or right toward the correct moving direction by sending vibration to the user's left or right footwear.

Heart rate sensor(s) can be added to the compass embedded footwear system to provide additional information about the user. The heart rate sensor should be properly connected with the control-communication unit for power, control, and communication, and be placed at a suitable position e.g., beneath the user's left and/or right sole. In various embodiments, the heart rate sensor can be used to detect whether a user is actually wearing the left and/or right footwear based on the detection of user heart beats. Using the information, the compass-embedded footwear system may automatically decide to switch to a power saving operation mode when there is no user that is actually wearing the footwear. In the power saving mode, the left/right footwear local processor 301 may read data only from the heart rate sensor in order to determine when to switch back to a normal operation mode when a user puts on the left/right footwear. This feature allowed by the heart rate sensor(s) may lead to significant saving on battery life and improve system life span.

In various embodiments, any combination of the bend sensor(s), motion sensor(s), GPS sensor(s), haptic component(s), and heart rate sensor(s) mentioned above, and/or any other types of sensors that may provide additional useful information can be added to the compass-embedded footwear system to support foot gesture detection and additional functions.

In some embodiments, the number of pressure sensors in a left/right footwear of the compass-embedded footwear system may be reduced to one. In this case, the footwear may detect whether a part of the footwear corresponding to the pressure sensor's positon is in touch to the ground. Such a configuration would provide much less information on user's foot action and support the detection of much less types of foot gestures. As a result, the disclosed functions for foot gesture detections cannot be fully supported by this configuration. However, it may significantly reduce the size of the left/right footwear. Optionally all the footwear components including the pressure sensor, the compass sensor, the control-communication unit, the battery module, and/or other suitable types of sensors such as a gyroscope, an accelerometer, a haptic component, etc., can be integrated into one single compact sole-attachable unit. Such a simplified configuration may allow the compass-embedded system to be easily switched among shoes, shoe insoles and easily work with any shoes regardless of shoe shape, size, owner, etc.

In other embodiments, the number of pressure sensors in a left/right footwear of the compass-embedded footwear system may be reduced to zero. In this case, the compass-embedded left/right footwear is not able to detect foot touches to the ground, while it still can provide foot directional information, i.e., V_{LF} and/or V_{RF} using measurements from the compass sensor. As a result, the fusion of left foot and right foot directional information may be less effective, and the types of foot gestures can be detected by the footwear system may be substantially reduced. However, removing all the pressure sensors may lead to a very compact design. Optionally, all the footwear components including the compass sensor, the control-communication unit, the battery module, and other useful types of sensors such as a gyroscope, an accelerometer, a haptic component, etc., can be integrated into one unit. And the placement of the integrated unit as the compass-embedded footwear may not need to be placed at a user's sole. For example, like the power-control-communication-compass unit, it can be placed on the outer surface of the footwear.

As such, there may be zero, one, two, three, four, five, six, seven, ten, hundred, and any number of pressure sensors in a single left/right footwear.

The compass embedded footwear system is an expandable system that supports functions including foot directional information extraction, user directional information extraction, foot gesture detections, and/or any other additional functions on top of it.

A systematic framework for user foot gesture detection based on user foot gesture features including user foot pointing direction(s) and foot touch state is described as follows.

In general, a system support user foot gesture detection and foot gesture based user-device control/interactions consists of i) a foot gesture feature information acquisition device, e.g., a compass-sensor embedded footwear system, used to obtain information related to foot gesture features and to distribute the information to an electronic device, and ii) an electronic device that receives information from the foot gesture feature information acquisition device, performing user foot gesture detections using the received information, and generate controls, including signals, message, etc. and perform operations based on foot gesture detection results.

Before the presenting the method for foot gesture detection, the definition of foot gestures is first presented. Pre-defined foot gestures may be stored in the storage media of the electronic device as data or as parts of executable codes to support the detection of the foot gestures.

In general, a (user) foot gesture is a sequence of foot gesture states. The length of the sequence can be any number from 1 to infinity.

Each foot gesture state corresponds to a set of requirements on foot gesture features. Different foot gesture states may have different requirements on the same set or different sets of foot gesture features. Foot gesture features includes (user) foot pointing direction related features, e.g., 701/702, (user) foot touch state, and others.

Note that the sequence of foot gesture states (or foot gesture state sequence) defining a foot gesture specifies i) a set of foot gesture states allowed by the foot gesture, and ii) a transition sequence formed by the (allowed) foot gesture states.

As an illustrative example, consider a first foot gesture given by a first abstract sequence of foot gesture state: S1->S2->S3. The foot gesture has three (allowed) foot gesture state, i.e., S1, S2 and S3, and requires the foot gesture state sequence to start from S1 then transit/switch to S2 then switch to S3. Here the -> mark is a sequential mark, which is used to connect two consecutive foot gesture states in the foot gesture state sequence.

A second foot gesture state sequence S2->S1->S3 has the same set of foot gesture states as S1, S2, S3. However, the transition sequence of the foot gestures states is different form that of the first foot gesture. As a result, the second foot gesture state sequence corresponds to a different foot gesture than the first foot gesture.

A foot gesture state sequence specifies a transition sequence formed by the (allowed) foot gesture states, which also infers that two consecutive foot gesture states cannot be the same, since there is no foot gesture state transition. For example, S1->S2->S2->S1 is not a valid foot gesture state sequence for a foot gesture, while S1->S2->S1 is a valid foot gesture state sequence.

Examples of various foot gesture definitions are given as follows.

Starting from the basics, various user foot gestures can be defined/detected based solely on the two basic types of foot gesture features, i.e., user foot pointing direction (s) and user foot touch state.

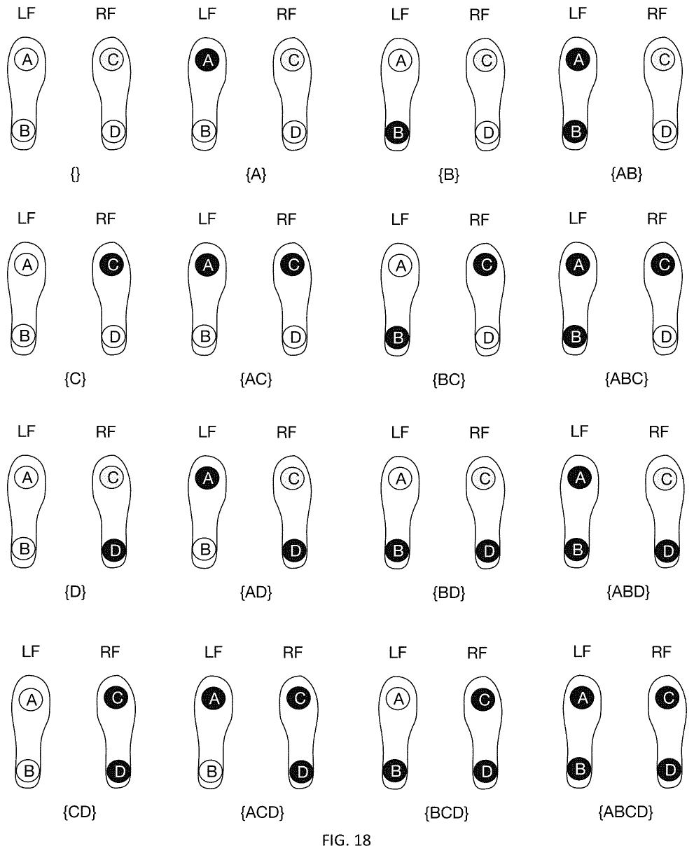

As earlier discussed, by processing information from the foot gesture feature information acquisition device, such as the compass-sensor embedded footwear system, foot touch state can be obtained. There are in total 16 Bi-foot touch states, which are shown in FIG. 18 with black circles indicating the corresponding foot areas are in touch with (or pressing) the ground. When only one Left or Right foot is involved, there are four single-foot touch states corresponding to left/right foot.

Conveniently the foot touch states are denoted as { }, {A}, {AB}, {ACD}, {ABCD}, etc., where A, B, C, and D in the bracket indicate foot sole areas touching the ground. Without loss of generality and following the definitions in FIG. 7, area A corresponds to a fore part of a user's left foot sole, area B corresponds to left foot heel, area C corresponds to a fore part of a user's right foot sole, and area D corresponds to right foot heel.

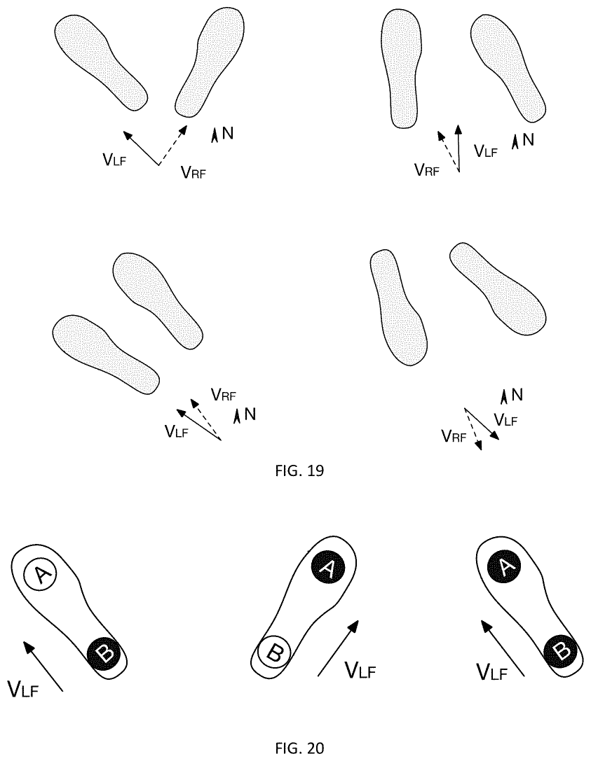

FIG. 19 illustrates left and right foot (2D) pointing directions, with foot pointing direction vectors V_{LF} (701) and V_{RF} (702) in reference to a local North direction. The foot pointing directions are obtained in a fixed/non-rotating 2D coordinate system, e.g., the user's local North-East coordinate system, that will not change with user's foot movements.

Foot gestures can be defined for single foot (single foot gestures) or both feet (Bi-foot gestures), which can be further categorized as touch based gestures, foot pointing direction based gestures, and combined gestures based on foot gesture features used for foot gesture detection.

Single Foot Gestures:

Single foot gestures are foot gestures based on actions of one foot. They are used when user foot pointing direction information and foot touch status is only available from one (either left of right) foot, e.g., when user only wears footwear on one foot. Single foot gestures can also be used for composing Bi-foot gestures.

Single Foot Touch Only Gestures:

Touch Based Single Foot Gestures can be defined and detected based on single (left/right) foot touch state.

Basic foot gestures may have a foot gesture state sequence with only one foot gesture state.

None in-touch foot gesture has a foot gesture state sequence of: { }.

The foot gesture state sequence has only one foot gesture state, i.e., { }, requiring left/right foot touch state to be { }.

Left foot full touch foot gesture has a foot gesture state sequence with only one foot gesture state, i.e., {AB}, requiring left foot touch state to be {AB}.

Right foot full touch foot gesture has a foot gesture state sequence with only one foot gesture state, i.e., {CD}, which requires right foot touch state to be {CD}

Left foot front only touch foot gesture has a foot gesture state sequence with only one foot gesture state, i.e., {A}, which requires left foot touch state to be {A}

Similarly defined, Right foot front only touch foot gesture has only one foot gesture state {C};

Left foot heel only touch foot gesture has only one foot gesture state {B}; and

Right foot heel only touch foot gesture has only one foot gesture state {D}.

A Left foot touch ground foot gesture has only one foot gesture state {A, B, AB}. Foot gesture state {A, B, AB} requires that left foot touch state to be {A} or {B} or {AB}, i.e., belonging to the set of left foot touch state {A, B, AB}. Here, notation {ele1, ele2, ele3} is used to denote the set of allowed foot touch states of the foot gesture state, where ele1, ele2, ele3, stands for foot touch state.

When the number of foot gesture states in the foot gesture state sequence is more than one, -> mark is used to connect two consecutive foot gesture states.

Type I Tap:

Description: foot heel part stays in touch with ground; fore part of a user's foot sole taps ground.

Left Foot Type I Tap gesture, denoted as LFTapI has the following foot gesture state sequence: {AB}->{B}->{AB}->{B}->{AB} . . .

The foot gesture has two (allowed) foot gesture states {B} and {AB}. Foot gesture state {B} requires that left foot touch state to be {B}. Foot gesture state {AB} requires that user right foot touch state to be {AB}.

Note that The foot gesture state sequence of LFTapI has an indefinite length and has a repetitive pattern.

Right Foot Type I Tap gesture, denoted as RFTapI, has the following foot gesture state sequence: {CD}->{D}->{CD}->{D}

The foot gesture has two allowed foot gestures states {C} and {CD}. Foot gesture state {C} requires that user right foot touch state to be {C}. Foot gesture state {CD} requires that user right foot touch state to be {CD}

The foot gesture state sequence has an indefinite length and a repetitive pattern.

A count parameter may be associated with a foot gesture that is repetitive in nature. For example, a foot gesture corresponds to the following foot gesture state sequence {AB}->{B}->{AB}->{B}->{AB}->{B}->{AB} which is a truncated version of foot gesture LFTapI. Such a foot gesture is denoted as LFTapI_3, where a count parameter 3 (connected to the notation of the corresponding un-truncated foot gesture, i.e., LFTapI, by a _ mark) indicating the number of repetitions of the foot gesture state pattern required by the foot gesture.

More generally LFTapI_n, (n=1, 2, 3, 4 . . . ) denotes a similar repetitive foot gesture (truncated) from foot gesture LFTapI, i.e., a finite length left foot type I Tap foot gesture. The count parameter n corresponds to the number of left foot Type I Taps required by the foot gesture.

Similarly, one has foot gestures RFTapI_n (n=0, 1, 2, 3 . . . ) which are finite length right foot type I Tap foot gestures. The count parameter n corresponds to the number of right foot Type I Taps required by the foot gesture.

Type II Tap:

Description: Fore part of a user's foot sole stays in touch with the ground; foot heel part taps ground.

Left Foot Type II Tap foot gesture, denoted as LFTapII, has the following foot gesture state sequence: {AB}->{A}->{AB}->{A} . . .

Similarly, LFTapII_n, (n=0, 1, 2, 3 . . . ) is a finite length Left Foot Type II Tap foot gesture, which has a foot gesture state sequence truncated from LFTapII. The count parameter n corresponds to the number of left foot Type II Taps required by the foot gesture.

Right Foot Type II Tap foot gesture, denoted as RFTapII, has the following foot gesture state sequence: {CD}->{D}->{CD}->{D} . . . Similarly, RFTapII_n, (n=1, 2, 3, 4 . . . ) is a finite length Rigth foot Type II Tap foot gesture, which has a foot gesture state sequence truncated from RFTapII. The count parameter n corresponds to the number of right foot Type II Taps required by the foot gesture.

Single Foot Step:

Description: foot alternatively leave and step on ground

Left foot Step, denoted as LFStep, has the following foot gesture state pattern: {A, AB, B}->{ }->{A, AB, B}->{ }->{A, AB, B} . . . The foot gesture has two (allowed) foot gesture states, i.e., {A, AB, B} and { }. Foot gesture state {A, AB, B} requires the user left foot touch state to be either {A} or {AB} or {B} and corresponds to user left foot touching the ground. And foot gesture sate { } requires the user left foot touch state to be { } and corresponds to user left foot left the ground. Note that foot gesture state {A, AB, B} consists of multiple foot touch states, as a result a change of foot touch state (as the foot gesture feature) does not necessarily change a foot gesture state.

Right foot Step, denoted as RFStep, has the following foot gesture state sequence: {C, CD, D}->{ }->{C, CD, D}->{ }->{C, CD, D} . . .

The foot gesture has two (allowed) foot gesture states, i.e., {C, CD, D} and { }. Both LFStep and RFStep foot gestures are repetitive in nature and have an indefinite foot gesture state sequence length. Similarly, finite length single foot step gestures can be denoted as: LFStep_n or RFStep_n (n=0, 1, 2, 3 . . . ), where count parameter n corresponds to the number of steps.

Note that gesture states such as {A, AB, B} and {C, CD, D} consist of multiple foot touch states. Change of user foot touch state will not change the foot gesture state as long as the foot touch states remain in the foot gesture state set. For a more concise notation of complex gesture state with multiple touch states, the | mark is used to represent an “or” relationship. For example, foot gesture state {A|B} requires that a user's foot touch state belongs to a set of all touch states with A or B foot sole area(s) touching the ground, which, for single foot touch only gesture is equivalent to {A, AB, B}. Accordingly, the following more concise notation for Left foot step gesture can be given as follows

Left foot Step (LFStep): {A|B}->{ }->{A|B}->{ }->{A|B} . . .

Right foot Step (RFStep): {C|D}->{ }->{C|D}->{ }->{C|D} . . .

Single Foot Direction Only Gestures

Foot gestures can be defined using only (2D) foot pointing direction related foot gesture features, such as foot pointing direction vector V_{LF}/V_{RF} (701/702) or foot pointing angle ω_{L}/ω_{R} (707/708). Two single foot gestures that use only 2D foot pointing direction related foot gesture features are Wiggle and Swipe.

A foot gesture state may have requirements on a foot pointing direction itself or on the change of foot pointing directions. Foot pointing angle information can be given in various forms, including a vector form V_{LF}/V_{RF} (701/702), or in the form of foot pointing angles ω_{L}/ω_{R} (707/708), which are all 2D foot pointing direction related foot gesture features. Requirements of a foot gesture state on a user foot pointing direction may be requiring the foot pointing direction vector V_{LF}/V_{RF} (701/702) to have clock wise rotation. Such requirements are denoted as by the foot pointing direction vector V_{LF} or V_{RF} followed by _R representing clockwise rotation, and the foot pointing direction vector V_{LF} or V_{RF} followed by L representing counter-clockwise rotation. Equivalently, when the foot pointing direction related foot gesture feature is a foot pointing angle ω_{L}/ω_{R} (707/708), the requirement of a foot gesture state on a foot pointing angle may be requiring a foot pointing angle ω_{L}/ω_{R} (707/708) to be in a value range. Such as a requirement can be denoted as {a<ω_{L}<b} or {a<ω_{L}}, with a specification of the foot pointing angle range enclosed by a pair of brackets. The requirement can also be requiring a foot pointing angle ω_{L}/ω_{R} (707/708) to increase or decrease, which can be denoted by the foot pointing angle ωL/ωR followed by _d representing a decreasing requirement, or followed by _u representing an increasing requirement, e.g., Ω_{L} _u, ω_{R} _d, etc.

Single Foot Wiggle Foot Gesture

Description: foot pointing direction rotates clockwise and counter-clockwise alternatively.

Left Foot Wiggle gesture, denoted as LFWig, has the following foot gesture state sequence:

V_{LF} _R->V_{LF}_L->V_{LF} R->V_{LF}_L . . . (Or equivalently, ω_{L} _u->ω_{L} _d->ω_{L} _u->ω_{L} _d . . . )

It has two foot gesture states, i.e., V_{LF} _R and V_{LF}_L. Foot gesture state V_{LF}_R requires left foot pointing direction to have clockwise rotation. Foot gesture state V_{LF}_L requires left foot pointing direction to have counter-clockwise rotation.

Right Foot Wiggle gesture, denoted as RFWig, has the following foot gesture state sequence: V_{RF} _R->V_{RF} _L->V_{RF} _R->V_{RF} _L . . .

It has two (allowed) foot gesture state V_{RF}_R and V_{RF} _L. Foot gesture state V_{RF} _R requires right foot pointing direction to have clockwise rotation. Foot gesture state V_{RF} _L requires right foot pointing direction to have counter-clockwise rotation.

Both LFWig and RFwig foot gestures are repetitive in nature and have indefinite foot gesture sequence length. Finite length left foot wiggle gesture and finite length right foot wiggle gestures can be denoted as: LFWig_n or RFWig_n (n=0, 1, 2, 3 . . . ), where count parameter n corresponds to the number of foot wiggle movements required by the foot gesture.

Single Foot Swipe Gesture:

Description: foot pointing direction rotates clockwise or counter-clockwise. A Swipe can be viewed as a half-Wiggle foot gesture.

Left Foot Swipe Left gesture, denoted by LFSwp_L has a foot gesture state sequence of length one, i.e., V_{LF} L.

It has one and only one foot gesture state V_{LF} _L requiring left foot pointing direction to perform counter-clockwise rotation. Left Foot Swipe Right gesture, denoted as LFSwp_R, has a foot gesture state sequence with only one foot gesture state: V_{LF} _R. The one foot gesture state V_{LF} _R requires left foot pointing direction to rotate clockwise.

Similarly, Right Foot Swipe Left gesture, denoted as RFSwp_L, has foot gesture state sequence: V_{RF} _L, which requires right foot pointing direction to rotate counter-clockwise. Right Foot Swipe Right gesture, denoted as RFSwp_R, has foot gesture state sequence: V_{RF} _R, which requires right foot pointing direction to rotate clockwise.

Using both user foot pointing direction and user foot touch state, more sophisticated foot gestures can be defined. Examples of single foot gestures using both types of the foot gesture features are described as follows.

Single Foot Directed Tapdown

Left foot directed Tap down Front, denoted as V_{LF} A, has a foot gesture state sequence containing only one foot gesture state: V_{LF}+{A}.

The one foot gesture state, denoted as V_{LF}+{A}, requires a user's left foot touch state to be {A} and left foot pointing direction is available from the foot gesture. Note that here the requirement on left foot pointing direction, e.g., in the form of V_{LF} is on its availability. The foot gesture requires user left foot pointing direction information, i.e., V_{LF} itself, to be provided when the foot gesture is detected. Also note that notation for the foot gesture state is V_{LF}+{A}, where requirements on two different foot gesture features are jointed by the + mark. The + mark is used represent the combination of multiple requirements of a foot gesture state on foot gesture features. And an availability requirement on foot gesture feature V_{LF} is denoted by putting the foot gesture feature's notation in the foot gesture feature notation as an independent requirement.

Left foot directed Tap down Heel, denoted as V_{LF} B, has a foot gesture state sequence containing only one foot gesture state V_{LF}+{B}. Foot gesture state V_{LF}+{B} requires that the user left foot touch state to be {B} and foot pointing direction information (in form of V_{LF}) needs to be available within the foot gesture. Note that V_{LF}+{B} is practically equivalent to ωL+{B}. And V_{LF}+{B} is the same as {B}+V_{LF}, since the order of multiple requirements of a foot gesture state does not matter.

Right foot directed Tap down front, denoted as V_{RF}C, has a foot gesture state sequence with only one foot gesture state V_{RF}+{C}.

Right foot directed Tap down heel, denoted as V_{RF}D, has a foot gesture state sequence with only one foot gesture state V_{RF}+{D}.

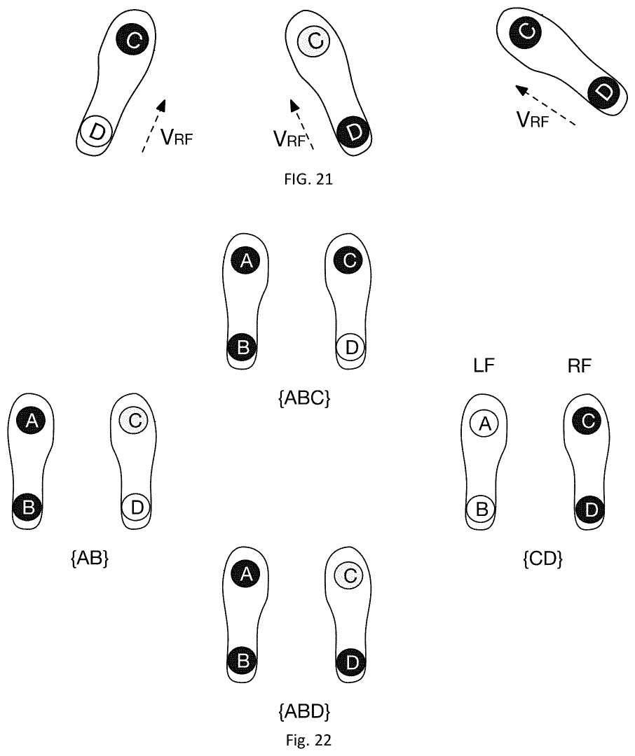

Comments: use single foot touch state to indicate forward (with only fore part of the sole touching ground) or backward (with only foot heel touching ground) movement along the foot pointing direction V_{LF} (the left foot pointing direction), or V_{RF} (the right foot pointing direction). Foot touch state {ABCD} can be used as a non-action state. In the following notations, + mark is used to denote a combination (simultaneous existence) of 2 or multiple element to form foot gestures. FIG. 20 illustrates left foot directed Tapdown gestures. FIG. 21 illustrates right foot directed Tapdown gestures.

Type I Wiggle

Description: foot Wiggle using foot heel as the pivot

Left foot type I Wiggle, denoted as LFWigB, has the following foot gesture state sequence: V_{LF} _R+{B}->V_{LF} _L+{B}->V_{LF} _R+{B}->V_{LF} _L+{B} . . . The foot gesture has two allowed foot gesture states, which are V_{LF} _R+{B} and V_{LF} _L+{B}. Foot gesture state V_{LF} _R+{B} requires that the foot touch state to be {B} and the foot pointing direction rotate clockwise. Foot gesture state V_{LF} _L+{B} requires that the foot touch state to be {B} and the foot pointing direction rotate counter-clockwise. The corresponding finite length left foot type I Wiggle foot gesture is denoted as LFWigB_n, where count parameter n denotes the number of wiggles performed.

Right foot type I Wiggle, denoted as RFWigD, has the following foot gesture state sequence: V_{RF} _D->V_{RF} _L+{D}->V_{RF} _R+{D}->V_{RF} _L+{D} . . . The corresponding finite length right foot type I Wiggle foot gesture is denoted as RFWigD_n, where count parameter n denotes the number of wiggles performed.

Type II Wiggle

Description: foot Wiggle using fore part of the foot sole as the pivot

Left foot type II Wiggle, denoted as LFWigA, has the following foot gesture state sequence: V_{LF} _R+{A}->V_{LF} _L+{A}->V_{LF} _R+{A}->V_{LF} _L+{A} . . .

The corresponding finite length left foot type II Wiggle foot gesture is denoted as LFWigA_n, where count parameter n denotes the number of wiggles performed.

Right foot type II Wiggle, denoted as RFWigC, has the following foot gesture state transition sequence:

V_{RF} _R+{C}->V_{RF}_R+{C}->V_{RF} _R+{C}->V_{RF}_L+{C} . . .

The corresponding finite length right foot type II Wiggle foot gesture is denoted as LFWigC_n, where count parameter n denotes the number of wiggles performed.

Single-Foot Type I Swipe Left: Foot Swipe Left Using Foot Heel as the Pivot

Left foot Type I Swipe Left has a foot gesture state sequence with only on foot gesture state V_{LF} L+{B}, which requires the left foot touch state to be {B} and left foot pointing direction rotate counter-clockwise V_{LF} _L.

Right foot Type I Swipe Left has a foot gesture state sequence with only on foot gesture state: V_{RF} _L+{D}

Single-foot Type II Swipe Left: foot Swipe left using fore part of the sole as the pivot

Left foot Type II Swipe Left has a foot gesture state sequence with only on foot gesture state: V_{LF} _L+{A}

Right foot Type II Swipe Left V_{RF} _L+{C}

Single-foot Type I Swipe Right: foot Swipe Right using foot heel as the pivot

Left foot Type I Swipe Right: V_{LF} _R+{B}

Right foot Type I Swipe Right: V_{RF} _R+{D}

Single-foot Type II Swipe Right: foot Swipe left using fore part of the sole as the pivot

Left foot Type II Swipe Right: V_{LF}_R+{A}

Right foot Type II Swipe Right: V_{RF} _R+{C}

Single foot gesture sequentially combined from basic single foot gestures:

[symbol]

Basic single foot gestures can be used as building blocks to construct new gestures. Sequentially combined foot gestures are still a sequence of foot gesture states. Such combined foot gestures are very useful for giving distinct control instructions to devices. Some examples of combined single gesture sequences are listed as follows. Similarly -> mark is used to connect two consecutive foot gestures, representing a sequential combination.

Finite length Type I Tap followed by a clock-wise Swipe

Description: left or right foot first performs Type I Tap gesture, which is followed by a clockwise swipe

For Left foot: LFTapI_n->LFSwp_R

For Right foot: RFTapI_n->RFSwp_R

Suggested use for device control: gesture for general program menu selection, gesture for zoom in/out (e.g., LFTapI_1->LFSwp_R as zoom out), etc.

Type I Tap followed by a counter-clock wise Swipe

Description: left or right foot first performs Type I Tap gesture, which is followed by a counter-clockwise swipe

For Left foot: LFTapI_n->LFSwp_L

For Right foot: RFTapI_n->RFSwp_L

Suggested use for device control: gesture for general program menu selection, gesture for zoom in/out (e.g., LFTapI_1->LFSwp_L as zoom in), etc.

Foot Gestures with Both Feet—Bi-foot gestures

Bi-Foot gestures with both of a user's feet involved are even more expressive. The possible set of Bi-foot gestures that can be constructed using foot gesture features including foot pointing directions and foot touch state is large. Customized gestures can be easily defined and detected using the disclosed methods. Here some examples of useful Bi-foot gestures are listed.

[symbol]

A set of touch-only Bi-Foot Gestures can be used to replace traditional arrow direction buttons for Left, Right, Up and Down, using touch only Bi-Foot gestures

Left: {AB}, or equivalently {AB}+{ }, when user shift whole weight to left foot

Right: {CD}, or equivalently { }+{CD}, when user shift whole weight to right foot

Up: {ABC, ACD} when user uses left or right foot's fore part of the sole to press the ground

Down: {ABD, BCD} when user uses left or right foot heel to press the ground

None: {ABCD} when user stands with all the four touch areas touching the ground.

As illustrated in FIG. 22, the Bi-Foot touch-only gesture set is capable of replacing the function of for direction buttons for up, down, left and right control.

[symbol]

The Bi-foot Directed Tapdown gestures use foot gesture features including Bi-foot touch state, and user foot pointing direction related foot gesture features, such as foot pointing direction vector V_{LF}/V_{RF}(701/702) or foot pointing angle ωL/ωR (707/708).

Given by their foot gesture state, Bi-foot Directed Tapdown gestures include:

V_{LF}+{ACD}, V_{LF}+{ABC}, V_{LF}+{BCD}, V_{RF}+{DAB}, etc.

Each Bi-foot Directed Tapdown gesture, e.g., V_{LF}+{ACD}, has a foot gesture state sequence containing only one foot gesture state. Foot gesture state V_{LF}+{ACD} requires that the user's Bi-foot touch state to be {ACD} and left foot (pointing) direction vector V_{LF} is provided with the foot gesture (which is an availability requirement on foot gesture feature V_{LF}). Other Bi-foot Directed Tapdown gestures are similarly defined.

Bi-foot Directed Tapdown gestures can be used to indicate movement along a direction given by left foot pointing direction V_{LF} or right foot pointing direction V_{RF}. For example

Forward movement in a direction: V_{LF}A+{CD} or V_{RF}C+{AB}

Backward movement in a direction: V_{LF}B+{CD} or V_{RF}D+{AB}

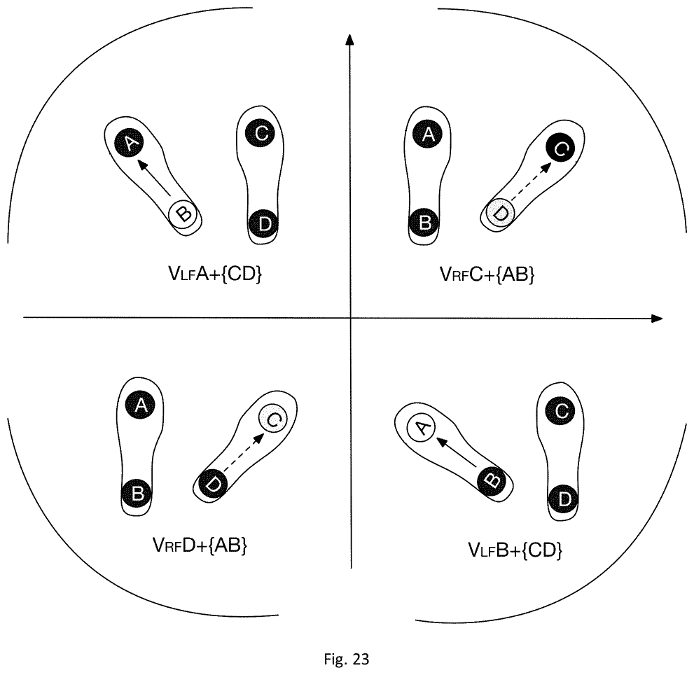

A set of Bi-Foot directed Tapdown gestures is capable of replacing the function of a joystick. One example of such a Bi-foot gesture set is shown in FIG. 23, where each Bi-foot gesture can be conveniently performed by user to provide direction control in one quarter of a fixed/non-rotating 2-D coordinate system.

Note that for Bi-foot gestures, when foot pointing direction information is used, it is often required to determine how to use the foot pointing direction from each foot. The decisions are made based on the left and right foot touch state. As in the Bi-foot directed Tapdown gestures, foot pointing direction of the foot that stays in full touch state is not used for direction control. This is an example that the joint use of foot touch state and foot pointing direction information is critical in the proposed foot gesture framework.

Walk as Bi-Foot Gesture

Description: user walk movement, with left and right foot alternatively leaving the ground

Walk foot gesture can be defined based on only Bi-foot touch state foot gesture feature. Walk foot gesture, denoted as Walk, has the following foot gesture state sequence:

{AB, A, B}->{ABCD,ABC,ABD,ACD,BCD,AC,BC,AD,BD}->{CD, C, D}->{ABCD,ABC,ABD,ACD,BCD,AC,BC,AD,BD}->{AB, A, B}->{ABCD,ABC,ABD,ACD,BCD,AC,BC,AD,BD}->{CD, C, D} . . .

It has three (allowed) foot gesture state, {AB, A, B}, {ABCD,ABC,ABD,ACD,BCD,AC,BC,AD,BD}, and {CD, C, D}.

Foot gesture state {AB, A, B} require Bi-foot touch state to be either {AB}, or {A} or {B} and corresponds to the user's left foot touching/pressing the ground and the user's right foot being up in the air.

Foot gesture state {ABCD,ABC,ABD,ACD,BCD,AC,BC,AD,BD} requires user Bi-foot touch state to be one of {ABCD}, {ABC}, {ABD}, {ACD}, {BCD}, {AC}, {BC}, {AD}, {BD}, corresponding to when user's both feet touching/pressing the ground.

Foot gesture state {CD, C, D} require Bi-foot touch state to be either {CD}, or {C} or {D} and corresponds to the user's right foot touching/pressing the ground and the user's left foot being up in the air

Additional parameter reflecting walking speed can be provided when foot moving trajectory state related foot gesture features are incorporated, which will be discussed later.

Note that a Bi-foot gesture state may consist of a set of large foot touch state as in the walk gesture. For more concise notations, a Bi-foot gesture state can be denoted as the combination of single left and right foot gesture states by connecting them with a + operator. For example, {ABCD,ABC,ABD,ACD,BCD,AC,BC,AD,BD} is equivalent to {A|B}+{C|D}, which means both left and right feet are in touch (or pressing) the ground. As introduced earlier, the operator represents a “or” relation ship, e.g., {A|B} is equivalent to {A, B, AB}. Walk as Bi-foot gesture has a gesture state sequence written with the concise notations as: {A|B}->{A|B}+{C|D}->{C|D}->{A|B}+{C|D}->{A|B} . . .

The corresponding finite Walk foot gestures, may be denoted as Walk_n (n=1, 2, 3, 4 . . . ), where n is a count parameter corresponding to walk steps.

Run as Bi-Foot Gesture

Description: user running movement, with left and right foot alternatively touching the ground

Denoted as Run, the foot gesture has foot gesture state sequence:

{A|B}+{ }->{ }->{ }+{C|D}->{ }->{A|B}+{ }->{ }->{ }+{C|D} . . .

The foot gesture has to three (allowed) foot gesture states, {A|B}+{ }, { } and { }+{C|D}. Foot gesture state {A|B}+{ } is a requirement on Bi-foot touch state formed by combining requirements on left foot touch state and on right foot touch state. It requires the left foot touch state to satisfy requirement {A|B}, i.e., left foot touches the ground, and, at the same time, the right foot touch state to satisfy requirement { }, i.e., right foot is untouched to the ground.

Similarly, Foot gesture state { }+{C|D} corresponds to the case when a user's left foot is untouched to the ground and, at the same time, user's right foot touch the ground. Foot gesture state { } requires Bi-foot touch state to be { }, corresponding to the case when user's both feet are in the air.

The corresponding finite length Run foot gesture is denoted as Run_n, (n=1, 2, 3 . . . ), where n is a step count requirement.

Additional parameter reflecting running speed can be provided when foot moving trajectory state related foot gesture features are added, which are discussed later.

Jump as Bi-Foot Gesture

Description: user jump movement with left and/or right feet touch the ground and both foot leave the ground simultaneously

Denoted as Jump, the foot gesture has the following foot gesture state sequence:

{A|B|C|D}->{ }->{A|B|C|D}->{ }->{A|B|C|D}->{ } . . . ,

It has two foot gestures states {A|B|C|D} and { }. Gesture state {A|B|C|D} requires the Bi-foot touch state to be one of a large set of 15 Bi-foot touch state that contains all foot touch states except the none touch state { }. It corresponds to the case when a user's either foot touches the ground or any supporting platform. The corresponding finite length Jump foot gestures are denoted by Jump_n (n=1, 2, 3 . . . ), where n is a jump count requirement.

Single-Footed Hop as Bi-Foot Gesture

Description: user jump on one leg

Left foot Single-footed hop gesture, denoted as LFHop, has the following foot gesture state sequence: {A|B}+{ }->{ }->{A|B}+{ }->{ }->{A|B}+{ }->{ } . . . It has two (allowed) foot gesture states, {A|B}+{ } and { }. Foot gesture state {A|B}+{ } specifies a requirement on a user's Bi-foot touch state, which corresponds to the case when a user's left foot touches the ground and a user's right foot is in the air.

The corresponding finite length Left foot Single-footed hop foot gestures are denoted by LFHop_n (n=1, 2, 3 . . . ), where n is the hop count

Right foot Single-footed hop gesture, denoted as RFHop, has a foot gesture state sequence as follows: { }+{C|D}->{ }->{ }+{C|D}->{ }->{ }+{C|D}->{ } . . .

It has two (allowed) foot gesture states, { }+{C|D} and { }.

Foot gesture state { }+{C|D} specifies a requirement on a user's Bi-foot touch state, which corresponds to the case when a user's right foot touches the ground and a user's left foot is in the air.

The corresponding finite length Right foot Single-footed hop foot gestures are denoted by RFHop_n (n=1, 2, 3 . . . ), where n is the number of hops specified by the foot gesture.

Note that, based on the foot gesture state sequences for Run_n, Jump_n, LFHop_n and RFHop_n, foot gesture Jump_n is a large foot gesture category containing Run_n, LFHop_n and RFHop_n, i.e., the detection of Run_n, LFHop_n or RFHop_n will lead to the detection of Jump_n. Jump_n also contains more sub foot gesture types such as Bi-foot jump. Bi-foot jump can be defined using the same gesture state sequence as Jump_n. However, it has an additional requirement in gesture state {A|B|C|D}. During foot gesture state {A|B|C|D}, the foot touch state need to enter/satisfy at least once a sub-gesture state {A|B}+{C|D} before leaving gesture state {A|B|C|D}, which enforces the requirement that both of the user feet need to be in touch with the ground at the same time during the {A|B|C|D} gesture state. In this case the sub gesture state {A|B}+{C|D} is considered as a landmark state of gesture state {A|B|C|D}. Requirement of landmark gesture state is added to a gesture state by a *{ } mark. For example {A|B|C|D}*{{A|B}+{C|D}} means gesture state {A|B|C|D} with landmark state {A|B}+{C|D}

Bi-Foot Jump as Bi-Foot Gesture

Description: user jump with both feet with left and right feet touch the ground and both foot leave the ground simultaneously

Bi-foot Jump foot gesture, denoted as BiJump, has the following foot gesture state sequence:

{A|B|C|D}*{{A|B}+{C|D}}->{ }->{A|B|C|D}*{{A|B}+{C|D}}->{ }->{A|B|C|D}*{{A|B}+{C|D}}->{ } . . .

It has two allowed foot gesture states {A|B|C|D}*{{A|B}+{C|D}} and { }. Foot gesture state {A|B|C|D}*{{A|B}+{C|D}} specifies requirements on a user's Bi-foot touch state. In the foot gesture state, the user's Bi-foot touch state must satisfy requirements from {A|B|C|D}, and before leaving the foot gesture state, e.g., to switch to the other foot gesture state { }, requirements from a sub-foot gesture state {A|B}+{C|D} must have been satisfied at least once. In general notation S1*{S2} denotes a foot gesture state defined by a foot gesture state S1 and a landmark foot gesture state S2, where the landmark state S2 is a sub-foot gesture state of S1 (which means foot gesture state S2 has stricter/more requirements than S1, i.e., when foot gesture features satisfy S2, they must also satisfy S1). Foot gesture state S1*{S2} requires that before leaving foot gesture state S1, the sub-foot gesture state S2 must be satisfied at least once, otherwise foot gesture state S1*{S2} is not deemed as being met.

The corresponding finite length BiJump foot gestures are denoted as BiJump_n (n=1, 2, 3, . . . ), where n is the number of Jumps specified by the foot gesture.

Incorporating foot pointing direction related foot gesture features to various walk, jump, hop gestures discussed above leads to more expressive foot gestures.

Directed Walk as Bi-Foot Gesture

Description: user walk movement, with left and right foot alternatively leaving the ground, while direction of each walking step is given based on the foot pointing direction or directions of user's feet, e.g., the supporting foot pointing direction

Directed Walk as Bi-foot gesture, denoted as VWalk, may have the following foot gesture state sequence:

{A|B}+{ }+V_{LF}->{A|B}+{C|D}->{ }+{C|D}+V_{RF}->{A|B}+{C}->{A|B}+{ }+V_{LF} . . .

The foot gesture has three foot gesture states {A|B}+{ }+V_{LF}, {A|B}+{C|D}, and { }+{C|D}+V_{RF}

Foot gesture state {A|B}+{ }+V_{LF} has requirements on foot gesture features given by {A|B}+{ }, and the availability requirement on left foot pointing direction foot gesture feature V_{LF}

Foot gesture state {A|B}+{C|D} has an requirement on foot gesture features given by {A|B}+{C|D}

Foot gesture state { }+{C|D}+V_{RF} has requirements on Bi-foot gesture state given by { }+{C|D}, and the availability requirement on right foot pointing direction foot gesture feature V_{RF}

The corresponding finite length VWalk foot gestures are denoted as VWalk_n (n=1, 2, 3 . . . ), where n is a count number specifying requirements on the number of walking steps.

Directed Run as Bi-Foot Gesture

Description: user running movement, with left and right foot alternatively touching the ground, while direction of each running step is given based on the foot pointing direction or directions of user's feet, e.g., the supporting foot pointing direction

Directed Run foot gesture, denoted as VRun, may have the following foot gesture state sequence: {A|B}+{ }+V_{LF}->{ }->{ }+{C|D}+V_{RF}->{ }->{A|B}+{ }+V_{LF}->{ }->{ }+{C|D}+V_{RF}

The corresponding finite length VRun foot gestures are denoted as VRun_n, (n=1, 2, 3 . . . ), where n specifies the number of running steps.

Directed Jump as Bi-Foot Gesture

Description: user jump movement with left and/or right feet touch the ground and both foot leave the ground simultaneously, while direction of each jump step is given based on the foot pointing direction or directions of user's feet

Directed Jump foot gesture, denoted as VJump, may have the following foot gesture state sequence:

{A|B|C|D}+V_{LF}+V_{RF}->{ }->{A|B|C|D}+V_{LF}+V_{RF}->{ }->{A|B|C|D}+V_{LF}+V_{RF}->{ } . . .

The corresponding finite length VJump foot gestures are denoted as VJump_n, (n=1, 2, 3 . . . ), where n specifies the number of jumping steps.

Note that the direction of each jump is evaluated at foot gesture state {A|B|C|D}+V_{LF}+V_{RF}. The evaluation of the jump direction is based on V_{LF}, V_{RF} as well as the Bi-foot touch states. For example, with touch state {AB} detected for gesture state {A|B|C|D}, V_{LF} may be used as the jump direction; with touch state {ABC} detected for gesture state {A|B|C|D}, the jump direction may be evaluated as a combination of V_{LF} and V_{RF}. This is another example where the joint use of foot pointing directions and touch state information is critical. Implementation for jump direction evaluation may vary for different applications, performance requirements or user preference.

Similarly, directed Single-footed hop and Bi-foot Jump foot gestures can be defined.

Directed Left foot Single-footed hop, denoted as VLHop, may have the following foot gesture state sequence: V_{LF}+{A|B}+{ }->{ }->V_{LF}+{A|B}+{ }->{ }->V_{LF}+{A|B}+{ }->{ } . . .

The corresponding finite length VLHop foot gestures are denoted as VLHop_n, (n=1, 2, 3 . . . ), where n specifies the number of hop count.

Directed Right foot Single-footed hop, denoted as VRHop, may have the following foot gesture state sequence V_{RF}+{ }+{C|D}->{ }->V_{RF}+{ }+{C|D}->{ }->V_{RF}+{ }+{C|D}->{ } . . .

The corresponding finite length VRHop foot gestures are denoted as VRHop_n, (n=1, 2, 3 . . . ), where n specifies the number of hop count.

Directed Bi-foot Jump foot gesture, denoted as VBiJump, may have the following foot gesture state sequence: {A|B|C|D}*{{A|B}+{C|D}}+V_{LF}+V_{RF}->{ }->A|B|C|D*{{A|B}+{C|D}}+V_{LF}+V_{RF}->{ }->{A|B|C|D}*{{A|B}+{C|D}}+V_{LF}+V_{RF}—>{ } . . .

The corresponding finite length VBiJump foot gestures are denoted as VBiJump_n, (n=1, 2, 3 . . . ), where n specifies the number of jump count.

Similar to VJump_n, the evaluation of the jump direction at each jump is done based on foot gesture features V_{LF}, V_{RF}, and foot touch states. Detailed implementation may vary for different applications, performance requirements or user preference.

Besides foot pointing direction related foot gesture features and foot touch state, user foot tilt angle γ_{L}/γ_{R} (1001/1002) can also be used as a foot gesture feature for foot gesture definition and detection. For tapDown or directed tapDown foot gestures, foot tilt angle can be used as additional parameters. FIG. 25 illustrates the foot tilt angles associated with Tapdown or directed Tapdown foot gestures.

As illustrated in FIG. 25, in a Tapdown or directed Tapdown foot gesture, the user's foot sole is not level to the ground. To characterize the tilted foot position, a user foot tilt γ_{L}/γ_{R} (1001/1002) for left/right foot can be used, which is the angle from the middle line of the foot sole along the sole surface (1003) to a leveled ground surface.

As an additional type of foot gesture feature, foot tilt angles can be incorporated to single foot directed Tapdown foot gestures, including V_{LF}+{A}, V_{LF}+{B}, V_{RF}+{C} and V_{RF}+{D} in many applications to offer desired user experiences.

Such foot gestures with foot tilt angle each have one foot gesture state, e.g., V_{LF}+γ_{L}+{A}, V_{LF}+γ_{L}+{B}, V_{RF}+γ_{R}+{C} and V_{RF}+γ_{R}+{D}

Foot gesture state V_{LF}+n+{A}, which is also a foot gesture since the foot gesture has only one foot gesture state, requires the user's left foot touch state to be {A} and requires the that V_{LF} and γ_{L} are provided with the foot gesture.

For example, such directed Tapdown gestures with tilt angles can be used for movement control in a direction (e.g., to replace the function of a joystick as illustrated in FIG. 23). The tilt angle of the corresponding foot n/γ_{R} can be used as a control parameter (representing “strength” of the control) to indicate the speed of the movement in the intended direction (e.g., larger tilt angle for higher moving speed). Without the additional tilt angle, the directed Tapdown foot gestures can only be used to give instructions on whether a movement should be made in a direction.

Similarly, foot tilt angles can be used with touch only foot gestures, e.g., the set of touch only foot gestures illustrated in FIG. 22, to indicate “strength” of the control.

In another example, the directed Tapdown foot gesture, such as V_{RF}+{D} as shown in FIG. 27, can be used in car driving applications for acceleration and brake controls. A directed Tapdown foot gesture V_{RF}+{D} in one direction, as shown in FIG. 27a can be used for brake and a directed Tapdown foot gesture V_{RF}+{D} in another direction as shown in FIG. 27b can be used for acceleration. The additional foot tilt angle of the corresponding foot can be used as foot gesture “strength” information to indicate how hard the user is stepping on the acceleration or brake to deliver realistic user driving control experiences.

Also note that besides foot tilt angles γ_{L}/γ_{R} (1001/1002), pressure levels at sole areas A, B, C and D can be used as foot gesture features to indicate foot gesture “strength” or for definition and detection of new types of foot gestures.

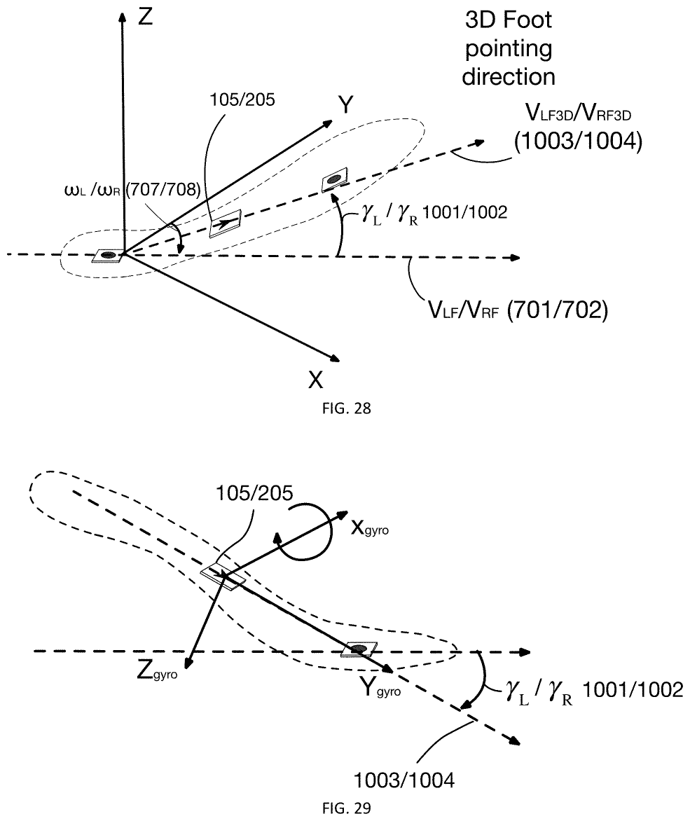

The introduction of foot tilt angles in fact generalizes the user foot pointing direction to a 3D space. FIG. 28 illustrates the relationships between foot tilt angle γL/γR (1001/1002), the original 2D foot pointing direction vector V_{LF}/V_{RF} (701/702) and a 3D foot pointing direction V_{LF}3D V_{RF}3D (1003/1004) in a local stationary (fixed/non-rotating) 3D coordinate system that does not rotate with user movement. The X-Y plane of the local 3D coordinate system is assumed to be level (or parallel to a leveled ground surface), the Z axis of the coordinate system is perpendicular to the X-Y plane. The 2D foot pointing direction is defined in the X-Y plane and can be specified by a foot pointing direction vector V_{LF}/V_{RF} (701/702). The 3D foot pointing direction vector V_{LF}3D/V_{RF}3D is the same as vector 1003/1004 in FIG. 25. It can be seen that V_{LF}/V_{RF} (701/702) is the projected direction vector of V_{LF}3D/V_{RF}3D (1003/1004) in the X-Y plane. While γ_{L}/γ_{R} (1001/1002) is defined in the plane formed by V_{LF}3D/V_{RF}3D (1003/1004) and the local Z axis, which is the elevation of the 3D foot pointing direction with respect to the X-Y plane.

In particular foot tilt angle can be used to define various Tap foot gestures. For example, single foot Type I Tap gestures can be redefined with foot tilt angle information.

Foot gesture Left foot Type I Tap with tilt, denoted as LFTapIwT, has the following foot gesture state sequence.

{B, AB}+γ_{L}_u+{0≤γ_{L}}->{B, AB}+γ_{L}_d+{0≤γ_{L}}->{B, AB}+γ_{L}_u+{0≤γ_{L}}->{B, AB}+γ_{L}_d+{0≤γ_{L}}->{B, AB}+γ_{L} _u+{0≤γ_{L}}->{B|AB}+γ_{L} _d+{0≤γ_{L}} . . .

It has two foot gesture states, i.e., {B|AB}+γ_{L}_u+{0≤γ_{L}} and {B|AB}+γ_{L}_d+{0≤γ_{L}}, with requirements on left foot touch state and left foot tilt angle.

Foot gesture state {B, AB}+γ_{L}_u+{0≤γ_{L}} has three requirements on foot gesture features. It requires that i) the foot touch state is either {B} or {AB}, ii) left foot tilt angle γ_{L} increase (such a requirement is denoted by γ_{L}_u), and iii) left foot tilt angle is non-negative, denoted as {0≤γ_{L}}.

Foot gesture state {B, AB}+γ_{L} _d+{0≤γ_{L}} has three requirements on foot gesture features. It requires that i) the foot touch state to be either {B} or {AB}, ii) left foot tilt angle γ_{L} to decrease (such a requirement is denoted by d), and iii) left foot tilt angle is non-negative, denoted as {0≤γ_{L}}.

Here, in the notation for each one of the three foot gesture states, three requirements on are types of foot gesture features are combined by + mark/operator.

The corresponding finite length Left foot Type I Tap with tilt is denoted as LFTapIwT_n, (n=1, 2, 3, 4, 5 . . . )

Similarly, foot gesture Left foot Type II Tap with tilt, denoted as LFTapIIwT, has the following foot gesture sate sequence.

{A, AB}+γ_{L}≤u+{γ_{L}≤0}->{A,AB}+γ_{L} _d+{γ_{L}≤0}->{A,AB}+γ_{L}≤u+{γ_{L}≤0}->{A,AB}+γ_{L}≤d+{γ_{L}≤0}->{A,AB}+γ_{L}≤u+{γ_{L}≤0}->{A,AB}+γ_{L}≤d+{γ_{L}≤0} . . .

It has two foot gesture states, {A, AB}+γ_{L}≤u+{γ_{L}≤0} and {A,AB}+γ_{L}≤d+{γ_{L}≤0}.

Foot gesture state {B, AB}+γ_{L} _u+{γ_{L}≤0} has three requirements on foot gesture features. It requires that i) the foot touch state is either {B} or {AB}, ii) left foot tilt angle γ_{L} increase (such a requirement is denoted by γ_{L}≤u), and iii) left foot tilt angle is non-positive, denoted as {γ_{L}≤0}.

Foot gesture state {B, AB}+γ_{L}≤d+{γ_{L}≤0} has three requirements on foot gesture features. It requires that i) the foot touch state to be either {B} or {AB}, ii) left foot tilt angle γ_{L} to decrease (such a requirement is denoted by γ_{L}_d), and iii) left foot tilt angle is non-positive, denoted by {γ_{L}≤0}.

The corresponding finite length Left foot Type II Tap with tilt is denoted as LFTapIIwT_n, (n=1, 2, 3, 4, 5 . . . ).

Similarly for right foot gesture, foot gesture right foot Type I Tap with tilt, denoted as RFTapIwT, has the following foot gesture state sequence.

{D, CD}+γ_{R} _u+{0≤γ_{R}}->{D, CD}+γ_{R} d+{0≤γ_{R}}->{D,CD}+γ_{L}≤u {0≤γ_{R}}->{D,CD}+γ_{R}≤d+{0γ_{R}}->{D,CD}+γ_{R} _u+{0≤γ_{R}}->{D,CD}+γ_{R} _d+{0≤γ_{R}} . . .

The corresponding finite Right foot Type I Tap with tilt foot gesture is denoted as RFTapIwT_n, (n=1, 2, 3, . . . )

Similarly, right foot Type II Tap with tilt can be defined as the following gesture state transition sequence with requirements on right foot touch states and right foot tilt angle.

{C|CD}+γ_{R} _u {γ_{R}≤0}->{C|CD}+γ_{R}≤d {γ_{R}≤0}->{C|CD}+γ_{R}≤u+{γ_{R}≤0}->{C|CD}+γ_{R}≤d+{γ_{R}≤0}->{C|CD}+γ_{R}≤u+{γ_{R}≤0}->{C|CD}+γ_{R}≤d+{γ_{R}≤0} . . .

The Right foot Type II Tap with tilt foot gesture is denoted as RFTapIIwT_n

Using also foot tilt angle as foot gesture feature, another category of foot gestures is 3D directed Tapdown which are extended from the (2D) directed Tapdown foot gestures as illustrated in FIGS. 20 and 21 with the additional foot tilt angle information. These 3D directed Tapdown foot gestures are foot gestures with only one foot gesture state, including

Left foot 3D directed Tapdown Front: V_{LF}+γ_{L}+{A}

Left foot 3D directed Tapdown Heel V_{LF}+γ_{L}+{B}

Right foot 3D directed Tapdown front: V_{RF}+γ_{R}+{C}

Right foot 3D directed Tapdown heel: V_{RF}+γ_{R}+{D}

Single foot 3D directed Tapdown gestures are denoted as V_{LF3D}A, V_{LF3D}B, V_{RF3D}C, and V_{RF3D}D respectively, which can be used in many applications for navigation direction control in a simulated 3D space.

Similarly, Bi-foot gestures, such as Bi-foot Directed Tapdown gestures shown in FIG. 23, can be extended with foot tilt angle foot gesture feature to 3D Bi-foot Directed Tapdown gestures for advanced navigation control in 2D or navigation in 3D. For example, Bi-foot directed Tapdown foot gestures V_{LF}A+{CD}, V_{RF} C+{AB}, V_{LF}B+{CD} and V_{RF} D+{AB} can be extended to form 3D directed Bi-foot Tapdown gestures V_{LF}+γ_{L}+{A}+{CD}, V_{RF}+γ_{R}+{C}+{AB}, V_{LF}+γ_{L}+{B}+{CD} and V_{RF}+γ_{R}+{D}+{AB}, which are denoted as V_{LF3D}A+{CD}, V_{RF3D}C+{AB}, V_{LF3D} B+{CD} and V_{RF3D}D+{AB} respectively.

From a 3D foot moving trajectory (1008/1009), as introduced earlier in the disclosure, many foot moving trajectory state related features can be extracted and used as foot gesture features. Without loss of generality, assume a foot moving trajectory with trajectory states VTraj[k1], VTraj[k1+1], VTraj[k1+2], . . . , VTraj[k2], k1<k2, associated with a foot gesture. Some examples of foot moving trajectory related features that can be extracted from the foot moving trajectory states are listed in the Table 1.

TABLE 1
A list of examples of foot moving trajectory sate related features

| | Foot moving | | |
|---|---|---|---|
| | trajectory | | |
| Notation | Features | Description | Example applications |
| Traj-1 | x[k2] , y[k2], | Position coordinates at the ending | Derive the direction of a |
| | z[k2] | time of the foot gesture | step in the fixed 3D or 2D |
| | | | (X-Y) coordinate system |
| Traj-2 | x[k3], y[k3], | Farthest (position) coordinates in | Derive kicking direction, |
| | z[k3] | the trajectory to the origin, where | derive foot traveled |
| | | k3 is the corresponding trajectory | distance in the foot |
| | | time index k1 ≤ k3 ≤ k2 | gesture |
| Traj-3 | vx[k4], | Velocity vector corresponding to | Used for early |
| | vy[k4], | the first peak speed in the foot | determination of |
| | vy[k4] | moving trajectory, where k4 is the | properties of a step or |
| | | corresponding trajectory time | jump foot gestures |
| | | index k1 ≤ k4 ≤ k2 | |
| Traj-4 | | 3D shape or projected shape in | May be used as foot |
| | | 2D of the foot moving trajectory. | gesture or composing |
| | | The shape can be triangle, circle, | combined foot gesture |
| | | V shape, L shape, etc. in the fixed | |
| | | 3D/2D coordinates | |
| Traj-5 | | Overall (average) angle between | May be used to |
| | | foot moving trajectory's 3D speed | distinguish different kick |
| | | vectors vx[k4], vy[k4], vy[k4] | style |
| | | and the 3D foot pointing | |
| | | directions (1003/1004) | |
| Traj-6 | | Total length of a foot moving | Use to determine foot |
| | | trajectory | moving distance and |
| | | | speed in some foot |
| | | | gestures |

A foot moving trajectory is mostly (but not always) associated with foot gestures that involves a period of time when a user's foot is in the air for example single foot gestures such as left/right foot steps, and Bi-foot gestures such as walk, run, and various jumps and their corresponding finite length foot gestures, e.g., Walk_n, Run_n, Jump_n, etc.

Left/right Foot moving trajectory states and various trajectory features derived from foot foot moving trajectory states are all foot moving trajectory related features that can be used as foot gesture features.

So far, key concepts for foot gesture detection, such as foot gesture, foot gesture state and foot gesture features, have been introduced. It is shown that various types of foot gestures, foot gesture states can be defined based on various foot gesture features including (2D) foot pointing direction, foot touch state, foot tilt angle, foot moving trajectory state related features, etc.

To summarize, a user foot gesture is a sequence of foot gesture states. Each foot gesture state corresponds to a set of requirements on a set of foot gesture features. The most important foot gesture features are user foot touch state and user 2D foot pointing direction(s) (which may be given in different various such as, foot pointing direction vector V_{LF}/V_{RF} (701/702) or foot pointing angle ω_{L}/ω_{R} (707/708)). Additional foot gesture features including foot tilt angle(s), foot roll angle(s), foot moving trajectory state related foot gesture features, etc.

Table 2 shows a list of foot gesture features, which is not an exhaustive list.

TABLE 2
A list of foot gesture features

| Foot gesture | | |
|---|---|---|
| feature name | Description | Examples |
| Foot touch | In some embodiments, Single-foot or Bi-foot | Single left foot touch |
| state (in some | touch state defined based on whether the fore part | state represented by |
| embodiments) | of a user's foot sole and the heel part of a user's | { }, {A}, {B}, {AB} |
| | foot sole touch/press the ground or a supporting | Single right foot touch |
| | platform. | state represented by |
| | There are 4 foot touch states for a single left/right | { }, {C}, {D}, {CD} |
| | foot and 16 Bi-foot touch states as shown in FIG. | Bi-foot touch states as |
| | 10 | shown in FIG. 18 |
| Foot touch | In other embodiments, foot touch state may be | |
| state (in other | determined based on the touching status of an | |
| embodiments) | arbitrary number, e.g., 1, 2, 3, of touch | |
| | points/areas on a user's foot sole. | |
| | In such cases, there may be more than 4 or less | |
| | than 4 single foot touch states, and there may be | |
| | more than 16 or less than 16 Bi-foot touch states. | |
| Foot pointing | A user foot pointing direction(s) is obtained in a | Foot direction vector |
| direction (2D | fixed/non-rotating 2D coordinate system, e.g., a | V_{LF}/V_{RF} (701/702), |
| foot pointing | user's local North East coordinate system. FIG. 8 | foot pointing angle |
| direction) | illustrates left/right foot pointing direction. Foot | ωL/ωR (707/708), |
| | pointing direction(s) can be given in various form, | fused user (forward) |
| | e.g., V_{LF}/V_{RF} (701/702) and ωL/ωR (707/708). | directional vector |
| | Such various forms of foot pointing direction are | V_{FWD} 709 |
| | also referred as foot pointing direction related foot | |
| | gesture features. | |
| Foot tilt angle | The tilt angle γ_{L}/γ_{R} (1001/1002) is illustrated in | foot tilt angle(s) γ_{L}/γ_{R} |
| | FIG. 25. | (1001/1002) |
| | Like foot pointing direction, foot tilt angle may be | |
| | given in various forms, which are all foot tilt | |
| | angle. | |
| Foot roll angle | Foot roll angle λ_{L}/λ_{R} (1005/1006) is illustrated | roll angle λ_{L}/λ_{R} |
| | in FIG. 26. | (1005/1006) |
| | Like foot pointing direction, foot roll angle may | |
| | be given in various forms, which are all foot roll | |
| | angle. | |
| Foot moving | 3D/2D user left/right foot moving trajectory | Foot moving |
| trajectory state | states, and various features derived using foot | trajectory related |
| related features | moving trajectory states. | features shown in |
| | | Table 1 |
| Foot 3D | User left/right foot acceleration, obtained from, | |
| acceleration | e.g., a 3-axis accelerometer, and/or derived foot | |
| | accelerations with coordinate conversion | |
| Foot angle | Obtained from, e.g., a 3-axis angle rate (gyro) | |
| rate(s) | sensor | |
| Pressure levels | Obtained by e.g., pressure sensors arranged under | Pressure levels (P_{A}, |
| at different foot | a user's foot sole(s) | P_{B})/(P_{C}, P_{D}) |
| sole areas | | |
| Time duration | Time duration of a foot gesture state from the | |
| of foot gesture | starting time of the foot gesture state can be easily | |
| state (s) | measured. | |
| | The time duration can be used as a foot gesture | |
| | feature. A foot gesture state may have requirement | |
| | on this foot gesture feature, i.e., the time duration | |
| | of the foot gesture state. For example, a foot | |
| | gesture state may require its duration to be less | |
| | than a threshold. | |
| Fused foot | Foot gesture features derived from other foot | V_{FWD} (701) as |
| gesture feature | gesture features | illustrated in FIG. 11 |

TABLE 3
A list of Key terminologies related to foot gestures

| Terminologies | Description | Examples |
|---|---|---|
| Foot gesture | Various types of user foot gestures, | Walk, Run, Tap, tapdown, |
| | actions. Each foot gesture corresponds | directed walk, directed |
| | to a sequence of foot gesture states | tapdown, etc. |
| Foot gesture | Various measurable (obtainable) | Foot pointing direction(s): |
| features | features that can be used for the | V_{LF}/V_{RF} (701/702) or ω_{L}/ω_{R} |
| | detection of foot gesture(s) | (707/708) |
| | | Foot touch state |
| | | Foot tilt angle(s): γ_{L}/γ_{R} |
| | | (1001/1002) |
| | | Foot moving trajectory state |
| | | related features |
| Information | Information that can be used to | Pressure measurements at |
| related to foot | derive/obtain foot gesture features, or | different sole areas, |
| gesture features | obtained foot gesture features | Measurements from compass |
| | | sensor, accelerometer, angle |
| | | rate sensor. |
| | | Various foot gesture features |
| Foot gesture | A device used for obtaining and | Compass-sensor embedded |
| feature | distributing information related to foot | footwear system |
| information | gesture feature(s). | |
| acquisition device | The acquisition device normally | |
| | consists of sensing components, local | |
| | processing components, and | |
| | communication components. | |
| | It can be one device or multiple | |
| | devices work jointly for the | |
| | monitoring of various foot gesture | |
| | features. | |

Table 3 lists key terminologies used in the present disclosure to clarify their meanings.

The foot gesture feature information acquisition device and its methods are summarized as follows.

A foot gesture feature information acquisition device, such as the compass-sensor embedded footwear system, is able to communicate with an electronic device. The foot gesture feature information acquisition device is able to acquire, at a data sampling or information acquisition time, information related to various foot gesture features including 2D foot pointing directions, (in a certain form, e.g., foot direction vector V_{LF}/V_{RF} (701/702) or foot pointing angle ω_{L}/ω_{R} (707/708)), fused user (forward) directional vector V_{FWD} 709, and foot touch state such as single foot touch states and/or Bi-foot touch states.

The foot gesture feature information acquisition device may also obtain information related to additional foot gesture feature including foot tilt angle(s) γ_{L}/γ_{R} (1001/1002) from a user's one foot or both feet.

The foot gesture feature information acquisition device may also obtain information related to additional foot gesture features including various foot moving trajectory state related features.

The acquired information related to foot gesture features at the sampling time is then sent to an electronic device through a communication link for foot gesture detections, which makes the foot gesture feature information acquisition device an input device. Note that the communication link may be an internal communication link when the electronic device is also a foot gesture feature information acquisition device. In some embodiments, such as in the compass-sensor embedded footwear system, multiple physically separated devices may work together as a foot gesture feature information acquisition device.

Before presenting the foot gesture detection method and device, a summary and further clarification of key concepts is given as follows.

A user foot gesture (including various single foot gestures and bi-foot gestures) corresponds to a (pre-defined) sequence of foot gesture states. The sequence of foot gesture states specifies i) a set of (allowed) foot gesture states of the foot gesture, and ii) required transitions of the foot gesture states. Length of the foot gesture state sequence could be 1 to infinity.

A foot gesture state corresponds to a set of requirements on a set of foot gesture features (see table 2). Each foot gesture state relates to a specific set of foot gesture features. For example, in Tap gestures, e.g., Type I Tap, their foot gesture states use (is related to) foot touch state; in single foot wiggle foot gestures, e.g., LFWig_n, the foot gesture states are related to foot pointing direction(s); and, in directed Tap down foot gestures, e.g., V_{LF} A, the foot gesture states are related to both foot pointing direction and foot touch state.

Requirements on foot gesture features of a foot gesture state can be given in various forms with some commonly used listed in Table 4.

TABLE 4
Types of requirements on foot gesture features of a foot gesture state

| Requirement type | Examples |
|---|---|
| Requiring foot gesture | Requiring foot pointing direction falls in a certain range, |
| feature value (if there is a | foot tilt angle falls in a certain range |
| value) falls in a certain | Foot gesture state {−5 < γ_{L} < 5} requires left foot tilt angle |
| range or ranges. | γ_{L} to be in an interval from −5 (degree) to 5 (degree) |
| | Requiring time duration of a foot gesture state to be lower |
| | than a threshold. |
| | (degree) |
| Requiring gesture feature | For example, requiring user foot touch state belong to a |
| belonging to a set. | foot touch state set. |
| | Foot gesture state {AB, A, B} requires user foot touch state |
| | to be either {AB} or {A} or {B} |
| Requirements on changes of | Requiring foot pointing direction, e.g., from left to right |
| foot gesture feature(s), e.g., | (clockwise), foot tilt angle increase, foot tilt angle decrease |
| requiring foot gesture | Example foot gesture states V_{LF}_L, V_{LF}_R, γ_{L}_u , γ_{L}_d |
| feature value to increase or | |
| decrease | |
| Requiring foot gesture | Adding a landmark gesture sub-state to a foot gesture state. |
| feature(s) to meet a | See foot gesture state {A\|B\|C\|D}*{{A\|B} + {C\|D}} of the |
| requirement(s) before | Bi-foot Jump foot gesture BiJump for an example. |
| leaving a foot gesture state. | |
| Availability requirement: | Foot gesture sate V_{LF} + {A} requires pointing direction |
| requiring a foot gesture | vector V_{LF} to be available |
| feature to be available | Foot gesture state V_{LF} + γ_{L} + {A}, require both foot pointing, |
| | direction vector V_{LF} (701) and foot tilt angle to be available |

The following foot gesture examples are used to further explain foot gesture, foot gesture states, and requirements on foot gesture features of a foot gesture state.

Foot Gesture Example 1

Left Foot Type I Tap has the Following Foot Gesture State Sequence

{AB}->{B}->{AB}->{B}->{AB} . . . The foot gesture consists of two foot gesture states, i.e., {AB} and {B}, which are defined by requirements on the foot gesture feature of (single/left foot) foot touch state. One foot gesture state has requirement {AB} requiring left foot touch state to be {AB}. The other foot gesture state has requirement {B} requiring left foot touch state to be {B}.

Foot Gesture Example 2

As another example, Walk as Bi-foot gesture is based on Bi-foot touch states and has a foot gesture state sequence as follows, {AB, A, B}->{ABCD,ABC,ABD,ACD,BCD,AC,BC,AD,BD}->{CD, C, D}->{ABCD,ABC,ABD,ACD,BCD,AC,BC,AD,BD}->{AB, A, B}->{ABCD,ABC,ABD,ACD,BCD,AC,BC,AD,BD}->{CD, C, D} or equivalently denoted as, {A|B}->{A|B}+{C|D}->{C|D}->{A|B}+{C|D}->{A|B}.

The foot gesture has three foot gesture states. The first gesture state is defined/given by requirements {AB, A, B} (or equivalently denoted as {A|B}) requiring Bi-foot touch state to be either {A} or {AB} or {B}, which corresponds to requiring a user's left foot touching the ground and user's right foot untouched to the ground.

The second foot gesture state is defined by requirement {ABCD,ABC,ABD,ACD,BCD,AC,BC,AD,BD} (equivalent to {A|B}+{C|D}) requiring Bi-foot touch state to be {ABCD} or {ABC} or {ABD} or {ACD} or {BCD} or {AC} or {BC} or {AD} or {BD}, which corresponds to requiring a user's left and right foot simultaneously touching the ground.

The third foot gesture state is defined by requirement {CD, C, D} (or equivalently {C|D}) requiring the Bi-foot touch state to be {C} or {D} or {CD}, which correspond to requiring a user's right foot touching the ground and left foot untouched to the ground.

The foot gesture state sequence defining the walk foot gesture also specifies the required transitions of the three foot gesture states. For example, a transition from foot gesture state {A|B} to foot gesture state {C|D}, i.e., {A|B}->{C|D}, is an un-allowed transition, which would violate the foot gesture state sequence and end the walk foot gesture.

Foot Gesture Example 3

As another example, based on foot gesture features including user left foot touch state and left foot tilt angle, a first foot gesture state may be defined by requirements {B, AB}+{-5<γ_{L}<5} requiring that the left foot touch state belongs to a set {B, AB}, and the left foot tilt angle falls in a interval between −5 degree and 5 degree. A second foot gesture state may be defined by requirements {A,AB}+{5≤γ_{L}≤15}, requiring that the left foot touch state belongs to a set {B, AB} and the left foot tilt angle falls in a interval between 5 degree and 15 degree. A third foot gesture state may be defined by {A,AB}+{15<γ_{L}} requiring that the left foot touch state belongs to a set {B, AB} and left foot tilt angle is greater than 15 degree.

Based on foot gesture features including user left foot touch state and left foot tilt angle, a type of a left foot Tapping foot gesture can be defined by a sequence of the three foot gesture states as {B, AB}+{-5<γ_{L}<5}->{A,AB}+{5≤γ_{L}≤15}->{A,AB}+{15<}->{A,AB}+{5≤γ_{L}≤15}->{B, AB}+{−5≤γ_{L}<5}->{A,AB}+{5≤γ_{L}<15}->{A,AB}+{15<γ_{L}} . . .

Foot Gesture Example 4

Based on the same set of foot gesture features as in example 3 i.e., left foot touch state and left foot tilt angle, another definition of a left foot Tapping foot gesture can be given by {{{B, AB}+{−5<γ_{L}<5} }|{{A,AB}+{5≤γ_{L}≤15}}}*{{B, AB}+{−5<γ_{L}<5} }->{{{A,AB}+{15<γ_{L}}}|{{A,AB}+{5≤γ_{L}≤15}}}*{{A,AB}+{15<γ_{L}}}->{{{B, AB}+{−5<γ_{L}<5}}|{{A,AB}+{5≤γ_{L}≤15}}}->}->{{{A,AB}+{15<γ_{L}}}|{{A,AB}+{5≤γ_{L}≤15}}}*{{A,AB}+{15≤γ_{L}}} which includes two gesture states.

One foot gesture state is defined by requirements {{{B, AB}+{-5<γ_{L}<5}}|{{A,AB}+{5≤γ_{L}≤15}}}*{{B, AB}+{-5<γ_{L}<5} }. It requires either requirements {{B, AB}+{−5<γ_{L}<5}} is satisfied or requirements {{A,AB}+{5≤γ_{L}≤15}} is satisfied. In addition the foot gesture state {{{B, AB}+{-5<γ_{L}<5}}|{{A,AB}+{5≤γ_{L}≤15}}}*{{B, A|3}+{-5<γ_{L}<5}} has a sub-foot gesture state {B, AB}+{-5<γ_{L}<5} as a landmark state, requiring the landmark sub-foot gesture state need to be met before leaving the foot gesture state.

The other foot gesture state is defined by requirements {{{A,AB}+{15<γ_{L}}}|{{A,AB}+{5≤γ_{L}≤15}}}*{{A,AB}+{15<γ_{L}}}. It requires either requirements {A,AB}+{15<γ_{L}} is satisfied or requirements {A,AB}+{5≤γ_{L}≤15} is satisfied. In addition the foot gesture state has a sub-foot gesture state {A,AB}+{15<γ_{L}} as a landmark state, requiring the landmark sub-foot gesture state needs to be met before leaving the foot gesture state.

The method and device supporting the detection of various foot gestures and foot gesture based device/application control and interactions are described as follows.

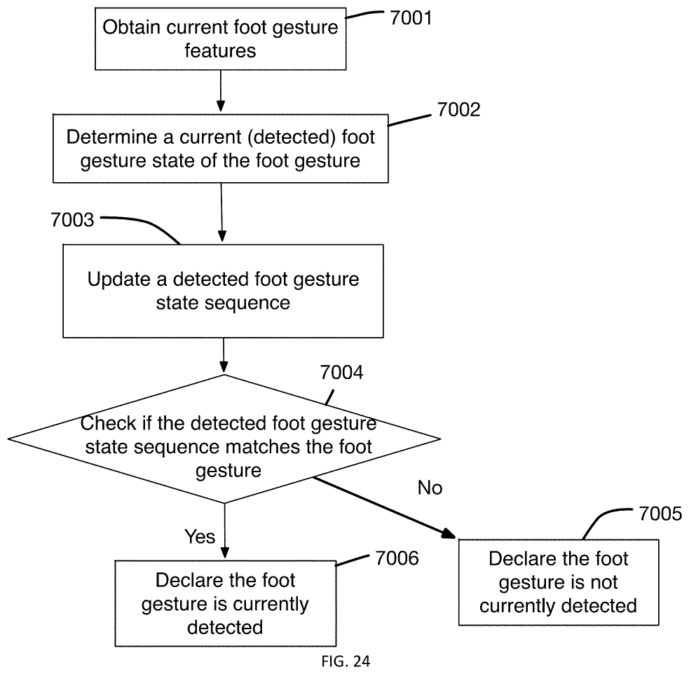

FIG. 24 shows the general processing flow for the detection of a pre-determined foot gesture which can be any foot gesture e.g., Walk, Run, Walk_n, LFHop, VWalk, LFWig_n, etc.

For foot gesture detection, information related to foot gesture features is provided by a foot gesture feature information acquisition device at data sampling/acquisition times. In some embodiments, the foot gesture detection process shown in FIG. 24 is executed at an electronic device by a foot gesture detector when information from the foot gesture feature information acquisition device at a new (current) sampling/acquisition time is received by an information receiver of the device. Note that, in some embodiments, there can be multiple foot gesture feature information acquisition devices simultaneously sending information related to foot gesture features to an electronic device.

In step 7001, current (updated) foot gesture features are obtained using the information form the foot gesture feature information acquisition device, e.g., a compass-sensor embedded footwear system. The foot gesture feature information acquisition device provides information related to foot gesture features, which may include foot gesture features and/or information that can be used to derive foot gesture features. In cases when a foot gesture feature needed for the detection of the pre-determined foot gesture is not directly available from the foot gesture feature information acquisition device, step 7001 involves the processing of received information to derive the missing foot gesture feature. Examples of such processing include the processing of pressure measurements to derive user foot touch state, and the processing of sensor measurements from the foot gesture feature information acquisition device to obtain foot moving trajectory state related foot gesture features.

Step 7002 uses the obtained current foot gesture features to determine a current (detected) foot gesture state of the pre-determined foot gesture. A foot gesture state is determined to be detected when all of its requirements on foot gesture features are met by the current foot gesture features. The result from step 7002 could be i) a (current) foot gesture state is detected, or ii) none of the pre-determined foot gesture's foot gesture states is detected.

Then based on the result from step 7002, step 7003 updates/maintains a sequence of detected foot gesture states, i.e., a detected foot gesture state sequence. Here an example is used to explain the detected foot gesture state sequence and the update done in step 7003.

With no loss of generality, assume that the pre-determined foot gesture has foot gesture states denoted as S1, S2, S3, S4, S5; and assume the foot gesture has a pre-determined foot gesture state sequence, e.g., S1->S2->S4->S3->S1->S5.

For the detection of the pre-determined foot gesture, a detected foot gesture state sequence is derived (updated) in step 7003, based on the detected foot gesture state of the foot gesture determined from step 7002 of the current and past processing rounds. If, at the current processing round, step 7002 has one of the foot gesture states detected, e.g., S1, which is different to the foot gesture state at the end of the detected foot gesture state sequence (corresponding to the latest foot gesture state in the detected foot gesture state sequence), the detected foot gesture state sequence is updated by adding the new detected foot gesture state, e.g., S1, to the end of the detected foot gesture state sequence as the latest foot gesture state in the sequence, which gives e.g., . . . ->S1.

If, at the current processing round, step 7002 has a detected foot gesture state, e.g, S1, which is the same as the detected foot gesture state found at the end of the detected foot gesture state sequence, there is no change in the detected foot gesture state sequence.

If, at the current processing round, step 7002 finds that none of the foot gesture states of the pre-determined foot gesture is detected, a Null state, denoted as Null, is detected and added to the end of the detected foot gesture state sequence, e.g., . . . ->S1->Null.

While the procedure for obtaining the detected foot gesture state sequence may vary. The detected foot gesture state sequence records transitions of the detected foot gesture states and detected Null states (corresponding to no foot gesture state of the foot gesture is detected) obtained from step 7002 of the current and history processing rounds.

With the updated detected foot gesture state sequence obtained from step 7003, step 7004 detects the pre-determined foot gesture by checking if the foot gesture state sequence of the pre-determined foot gesture is matched by the updated detected foot gesture state sequence from step 7003.

For a foot gesture whose foot gesture state sequence has a finite length N, a match requires that the last (latest) N states of the detected foot gesture state sequence match exactly the foot gesture sequence of the foot gesture.

Back to the previous example, the detected foot gesture state sequence may look like . . . S1->S2->S1->S5->Null->S2->S5. The foot gesture with foot gesture state sequence S1->S2->S4->S3->S1->S5 is only matched by the detected foot gesture state sequence (at the current processing round) when the updated detected foot gesture state sequence obtained in step 7003, looks like . . . ->S1->S2->S4->S3->S1->S5.

For a foot gesture with a foot gesture state sequence of an indefinite length. A match between the detected foot gesture state sequence and the foot gesture's foot gesture state sequence can be declared at any detected foot gesture state. A mismatch shall be declared whenever the portion of the detected foot gesture state sequence—starting from the state where the match is first declared to the latest state of the detected foot gesture state sequence—cannot be an exact match to any truncated segment of the foot gesture, i.e., violates the transition pattern of the foot gesture's foot gesture state sequence.

If, in step 7004, a match between the detected foot gesture state sequence and the foot gesture state sequence of the pre-determined foot gesture is detected, the processing flow moves on to step 7006 to declare the foot gesture is detected at the current time/processing round, which finishes the current round of processing.

If, in step 7004, a mismatch between the detected foot gesture state sequence and the foot gesture state sequence of the pre-determined foot gesture is detected, the processing flow moves on to step 7005 to declare the foot gesture is not detected at the current time/processing round, which finishes the current round of processing.

The foot gesture detection method according to the processing flow in FIG. 24 is generally implemented at a foot gesture detector of an electronic device to detect various user foot gestures.

The electronic device should have an information receiver, configured to receive information related to foot gesture features from an information acquisition device. In some embodiments, the electronic device can also be a foot gesture feature information acquisition device. In such cases, the information receiver receives information related to foot gesture features using internal communication in the electronic device.

When a user foot gesture is detected by the foot gesture detector of the electronic device, a command generator of the electronic device generates control signals corresponding to the detected foot gesture. Such control signals may take various forms including messages, foot gesture events, function calls, etc. The detected foot gesture may also provide various foot gesture features, such as user foot pointing direction, user foot tilt angle, etc., which may be part of the control signals. The control signals can be used by operating system of the electronic device to support foot gesture based device control and use-device interactions. The control signals can also be sent to various software programs, applications running on the electronic device to support foot gesture based application control and user-application interactions. The control signals can also be sent to other electronic device(s) through a communication link for the control of the electronic device and/or applications running on the device.

Applications

A foot gesture feature information acquisition device as an input device can be used as a foot-operated controller to support foot gesture based device/application control and user-device/user-application interactions. Some promising application areas are discussed below.

A foot operated controller, e.g., the compass embedded footwear system, can be used as complementary input device to hand operated control devices.

It can be use with game console controllers for new and enhanced user experience. Gaming applications using conventional game controllers with joysticks and direction buttons tends to overload user hands with control tasks. The foot operated controller allows the use of foot gestures to replace the functions of the joystick or the set of direction buttons, which effectively reduce control tasks on user hands. User's body weight shifts and various foot gestures can be used for game controls to provide more engaging gaming experiences.

A foot operated controller, e.g., the compass embedded footwear system, can be used with mobile device and applications for new and enhanced user experiences. When using a mobile device such as a smart phone or a tablet, a user's hands need to first hold the device, which makes it challenging to use fingers for device and application control. The foot operated controller allows a user to use rich foot gestures to interact with mobile devices and applications and offer user experiences that are more realistic and natural.

A foot operated controller, e.g., the compass embedded footwear system, can be used for handfree control in e.g., Kinect applications. Handfree device and application control is desirable in many applications to provide more relaxing user experiences. With user's hand freed from holding a controller, a user also has more freedom in using hand gestures to interact with applications. One example of handfree control is Kinect for Xbox. However, the video based hand and body gesture recognition has limitations in hand gesture detection speed. In addition, Kinect is lack in capability for foot gesture detection and requires the user to face the Kinect camera at all time. The foot operated controller offering valuable information from user's feet (foot gesture information, foot pointing direction information, etc.) and can be a great complementary control device to Kinect for improved and novel user experiences.

A foot-operated controller is essential to applications that need foot pointing direction information and foot gestures that are unavailable from hand operated control devices. In real world there are many cases when people's feet are used for control purposes. In car driving, the driver uses right foot to step on accelerator pedal and brake for acceleration and deceleration. In ski and skating, main controls come from foot actions and foot pointing directions instead of hands. In tennis games, a player's footwork is a crucial part of his/her play. Using user foot gestures and foot pointing direction information from the compass embedded footwear system, realistic user experiences can be achieved in applications including ski, skating, tennis, soccer, driving games. Some application ideas are listed as follows

Ski games: Foot pointing directions from the foot-operated controller allows the simulation of realistic ski board, ground surface, gravity interactions for realistic gaming experience.

Tennis games: With the foot-operated controller, user foot gestures such as Bi-foot Directed Tapdown, Directed Walk, Directed Run, Walk/Run with Trajectory feature etc., can be used for court movement control. Foot touch state information and foot pointing direction information can be used to form new foot gestures for user footwork detection.

Soccer games: A foot-operated controller allows the use of various foot gestures for movement control. Information from the foot-operated controller can be used for the detection of kicking direction and differentiate different passing and shooting techniques. Foot gestures can be used in such applications include, Single foot step gestures with foot moving trajectory feature, e.g., (LFStep+Traj-1)_n, (RFStep+Traj-1)_n, (LFStep+Traj-2+Traj-5)_n, (RFStep+Traj-2+Traj-5)_n, etc., and various Bi-foot gestures with foot moving trajectory features.

Driving games: A foot-operated controller allows the use of foot gestures such as Right Foot Directed Tapdowns or Directed Tapdown with tilt information for realistic acceleration and deceleration control.

A foot-operated controller is able to map user turns and intended moving direction in real world to virtual world to support realistic virtual world navigation.

In conventional control scheme for navigation control in a virtual world, in general, a user's moving direction is given in reference to the user's viewing direction. As a result, the change in a user's viewing direction also changes the user's moving direction.

However, in the real world, a person's viewing direction and a person's moving direction are independently controlled. One's viewing direction is controlled by his/her neck; and one's moving direction is independent to the viewing direction, which in general roughly aligns with his/her foot pointing directions. In real world, a person can move towards a direction and look around without changing the moving direction. However, such an everyday experience cannot be reproduced in a virtual world with the conventional scheme for navigation control.

A foot-operated controller is able to provide user foot pointing direction information in a fixed coordinate system that does not change with user movement, which can be further mapped to a fixed coordinate system in a virtual world. The foot pointing direction information can be naturally used to derive the user's intended moving direction, which effectively maps a user's turn movements in real world to turns in the virtual world.

It is particularly useful when a user uses a mobile device or wearing a video goggle, since the user can easily make turns. With the foot operated controller providing independent control for user's moving direction, real life viewing experiences such as moving in a direction while looking around or sideways can be achieved.

1. (canceled)

2. (canceled)

3. (canceled)

4. (canceled)

5. (canceled)

6. A method, comprising:
receiving information related to foot gesture features from an information acquisition device;
obtaining the foot gesture features using the received information;
detecting a foot gesture using the obtained foot gesture features; and
generating a control signal based on the detected foot gesture.

7. The method of claim 6, wherein:
a foot gesture includes a sequence of foot gesture states, and
each foot gesture state includes a set of requirements on the foot gesture features corresponding to the foot gesture.

8. The method of claim 7, wherein detecting the foot gesture using the obtained foot gesture features comprises:
determining a foot gesture state of the foot gesture as being currently detected when the obtained foot gesture features meet the set of requirements of the foot gesture state;
determining the foot gesture is currently detected, when a sequence formed by the detected foot gesture states matches the sequence of foot gesture states of the foot gesture; and
declaring the foot gesture is currently undetected, if the sequence formed by the detected foot gesture states does not match the sequence of foot gesture states of the foot gesture.

9. The method of claim 6, wherein the foot gesture features include:
a foot pointing direction of each of a user's one foot or both feet, and/or
a foot touch state of each of a user's one foot or both feet,
wherein the foot touch state is determined based on whether one or multiple parts of a user's foot sole touch or press a supporting platform, the supporting platform including the ground.

10. The method of claim 9, wherein the foot touch state is based on whether a fore part and a heel part of a user's foot sole touch or press a supporting platform, the supporting platform including the ground.

11. The method of claim 9, wherein the foot gesture features further include a foot tilt angle of each of a user's one foot or both feet.

12. The method of claim 9, wherein the foot gesture features further include foot moving trajectory state related features for a user's one foot or both feet.

13. The method of claim 6, further including:
processing to obtain the foot gesture features, when the received information from the information acquisition device is indirectly related to the foot gesture features.

14. An information acquisition device, comprising:
an information acquisition member configured to acquire information related to foot gesture features, the foot gesture features including one or more of a foot pointing direction of each of a user's one foot or both feet, a foot touch state of a of user's one foot, and a foot touch state of a user's both feet, wherein the foot touch state is determined based on whether one or multiple parts of a user's foot sole touch or press a supporting platform, the supporting platform including the ground; and
a communication member configured to send the acquired information related to the foot gesture features to an electronic device for a foot gesture detection.

15. The device of claim 14, wherein the foot gesture features further include one or more of a foot tilt angle of each of a user's one foot and both feet and foot moving trajectory state related features for a user's one foot or both feet.

16. The device of claim 14, wherein the information acquisition member includes a compass sensor assembly, the compass sensor assembly including a compass sensor, an accelerometer, an angle-rate sensor, physically combined as one platform.

17. The device of claim 16, wherein the one platform is placed at a position corresponding to a middle section of a user's foot in order to obtain a foot tilt angle in the foot touch states.

18. The device of claim 16, wherein the information acquisition member further includes one or more pressure sensors for obtaining the foot touch state.

19. The device of claim 18, wherein the one or more pressure sensors are placed at positions corresponding to the fore part of a user's foot sole and a heel part of a user's foot sole in order to determine if the corresponding part of a user's foot touches or presses a supporting platform including the ground.

20. The device of claim 14, further including a foot wearable device.

21. (canceled)

22. (canceled)

23. (canceled)

24. (canceled)

25. (canceled)

26. (canceled)

27. (canceled)

28. (canceled)

29. A non-transitory computer readable storage medium, storing computer-executable instructions for, when being executed, one or more processors to perform a method, the method comprising:
receiving information related to foot gesture features from an information acquisition device;
obtaining the foot gesture features using the received information;
detecting a foot gesture using the obtained foot gesture features; and
generating a control signal based on the detected foot gesture.

30. The method of claim 29, wherein:
a foot gesture includes a sequence of foot gesture states, and
each foot gesture state includes a set of requirements on the foot gesture features corresponding to the foot gesture.

31. The method of claim 30, wherein detecting the foot gesture using the obtained foot gesture features comprises:
determining a foot gesture state of the foot gesture as being currently detected when the obtained foot gesture features meet the set of requirements of the foot gesture state;
determining the foot gesture is currently detected, when a sequence formed by the detected foot gesture states matches the sequence of foot gesture states of the foot gesture; and
declaring the foot gesture is currently undetected, if the sequence formed by the detected foot gesture states does not match the sequence of foot gesture states of the foot gesture.

32. The method of claim 29, wherein the foot gesture features include:
a foot pointing direction of each of a user's one foot or both feet,
a foot touch state of each of a user's one foot or both feet,
a foot tilt angle of each of a user's one foot or both feet, and/or
foot moving trajectory state related features for a user's one foot or both feet,
wherein the foot touch state is determined based on whether one or multiple parts of a user's foot sole touch or press a supporting platform, the supporting platform including the ground.

33. The method of claim 32, wherein the foot touch state is based on whether a fore part and a heel part of a user's foot sole touch or press a supporting platform, the supporting platform including the ground.
